(19) United States
(12) Patent Application Publication
(10) Pub. No.: US 2010/0272787 A1
(43) Pub. Date: Oct. 28, 2010

(54) AMETHOD OF TREATING NEURODEGENERATIVE DISEASES

(75) Inventor: Alon Monsonego, Moshav Nir-Banim (IL)

Correspondence Address: MARTIN D. MOYNIHAN d/b/a PRTSI, INC., P.O. BOX 16446, ARLINGTON, VA, 22215, US

(73) Assignee: Ben Gurion University of the Negev, Beer-Sheva (IL)

(21) Appl. No.: 12/809,669

(22) PCT Filed: Dec. 18, 2008
(86) PCT No.: PCT/IL08/01639

§ 371 Date: Jun. 21, 2010

Related U.S. Application Data
(60) Provisional application No. 61/008,580, filed on Dec. 21, 2007.

(51) Int. Cl. A61K 39/395; A61K 38/21; A61K 9/127; A61P 25/28; A61P 29/00; A61P 25/16; A61P 25/02; A61P 25/00; A61K 31/7088; A61K 35/12

(52) U.S. Cl. 424/450; 424/85.5; 424/173.1; 514/44 R; 424/93.71

(57) ABSTRACT

This invention provides compositions and methods for treating a subject suffering from a disease or disorder of the nervous system, associated with an inflammatory response. This invention further provides a pharmaceutical composition comprising, inter alia, an agent which increases brain levels of interferon-γ; and an agent which reduces the number of brain T regulatory (Treg) cells, and optionally further comprising an agent which suppresses neurotoxic inflammatory brain responses.

### FIELD OF INVENTION

This invention is directed to compositions and treatment methods of subject suffering from a disease or disorder of the nervous system, associated with an inflammatory response.

### BACKGROUND OF THE INVENTION

Renewal of cells and their replenishment by new growth normally provides for tissue repair in most tissues of the body. The central nervous system (CNS) is particularly vulnerable to insults that result in cell death or damage in part because cells of the CNS have a limited capacity for repair. In the brain, especially, any loss of neurons results in functional deficits ranging from minor to devastating. Since damaged brain tissue does not regenerate, recovery must come from the remaining intact brain. Since an insult to the CNS, whether acute or chronic, is often followed by the post-injury spread of neuronal damage, much research has been devoted to finding ways to minimize this secondary degeneration by rescuing as many neurons as possible.

Under normal conditions in the adult brain, new neurons are formed in the neurogenic niches of the subventricular zone of the lateral ventricles and the subgranular zone of the hippocampal dentate gyrus. While many of these newborn cells die shortly after their birth, a number become functionally integrated into the surrounding brain tissue. Under pathological conditions some neurogenesis can also be induced in non-neurogenic brain areas. The function of adult neurogenesis is not certain, although there is some evidence that hippocampal adult neurogenesis is important for learning and memory. Experiments aimed at knocking out neurogenesis have proven inconclusive, although the overall findings that adult neurogenesis is important for any kind of learning are equivocal.

Under pathological conditions (when cell renewal is critical), microglia not only do not favor cell renewal, but interfere with it. As a result, activated microglia have generally been viewed as a uniformly hostile cell population that causes inflammation, interferes with cell survival and blocks cell renewal. This situation may be remedied by well-controlled adaptive immunity, which shapes the microglia in such a way that their activity is not cytotoxic, but is both protective and conducive to renewal. This suggests that both neurogenesis and gliogenesis are likely to occur in situations in which protective autoimmunity leads to improved recovery. These data can also explain the lack of cell renewal in autoimmune diseases; in such cases, it is likely that the number of circulating autoimmune T cells does not enable the microglia to acquire a protective phenotype. This would also explain why steroids are not helpful, as their anti-inflammatory activity masks not only the destructive, but also the beneficial adaptive immunity. The therapy of choice for both autoimmune diseases and neurodegenerative conditions would therefore appear to be immunomodulation in which, after the acute phase of disease, the surviving tissue can be maintained by relatively small quantities of T cells.

Poor recovery from acute insults or chronic degenerative disorders in the CNS has been attributed to lack of neurogenesis, limited regeneration of injured nerves, and extreme vulnerability to degenerative conditions. The absence of neurogenesis has been explained by the assumption that soon after birth the CNS reaches a permanently stable state, needed to maintain the equilibrium of the brain's complex tissue network. However, research over the last decade has shown that the brain is capable of generating new neurons and glia from precursor stem cells (neurogenesis) throughout life, albeit to a limited extent. However, new neurons generated in the dentate gyrus (DG) decline sharply with age, and to an even greater extent in neurodegenerative diseases.

Neurogenesis is blocked in the inflamed brain, strengthening the traditional view that local immune cells in the CNS have an adverse effect on neurogenesis. Likewise, the limited regeneration and excessive vulnerability of CNS neurons under inflammatory conditions or after an acute insult have been attributed to the poor ability of the CNS to tolerate the immune-derived defensive activity that is often associated with local inflammation and cytotoxicity mediated, for example, by tumor necrosis factor (TNF)-α or nitric oxide. More recent studies, however, have shown that although an uncontrolled local immune response indeed impairs neuronal survival and blocks repair processes, a local immune response that is well controlled with respect to the onset, duration and intensity by peripheral adaptive immune processes (in which CD4+ helper T cells are directed against self-antigens residing at the site of the lesion) can support neuronal survival after CNS injury and promote recovery. In addition, the proliferation and differentiation of neural progenitor cells (NPC) are significantly suppressed, primarily in the DG, with aging. This process may be related, at least partially, to the gradual advancement of cognitive deficits in the elderly. The induction of neurogenesis is of particular interest in the study of Alzheimer's disease (AD), since the pathology of AD occurs in brain regions of learning and memory, and there is, at present, no cure for the disease. Neurogenesis has been shown to be increased in the brains of patients with AD compared with age-matched control subjects, while different mouse models of AD have shown either reduced or enhanced neurogenesis in the DG. There is thus a clear need to clarify the factors supporting neurogenesis.

Neurogenesis occurs throughout life in adult individuals, albeit to a limited extent. Most of the newly formed cells die within the first 2-3 weeks after proliferation and only a few survive as mature neurons. Little is known about the mechanism(s) underlying the existence of neural stem/progenitor cells (NPCs) in an adult brain and why these cells are restricted in amount and limited to certain areas. Moreover, very little is known about how neurogenesis from an endogenous NPC pool can be physiologically increased. Knowledge of the factors allowing such stem cells to exist, proliferate and differentiate in the adult individual is a prerequisite for understanding and promoting the conditions conducive to CNS repair. This in turn can be expected to lead to the development of interventions aimed at boosting neural cell renewal from the endogenous stem cell pool or from exogenously applied stem cells.

### SUMMARY OF THE INVENTION

This invention provides, in one embodiment, a composition for inducing and/or enhancing neurogenesis in a subject, comprising:
a) an agent which increases brain levels of interferon-γ; and
b) an agent which reduces the number of brain T regulatory (Treg) cells.

In another embodiment, the composition further comprises an agent which suppresses neurotoxic inflammatory brain responses.

In one embodiment, the composition is formulated for brain-specific delivery. In another embodiment, the composition comprises a liposome.

In one embodiment, the composition may further comprise a carrier, diluent, lubricant, flow-aid, or a mixture thereof. In another embodiment, the composition is in the form of a pellet, a tablet, a capsule, a solution, a suspension, a dispersion, an emulsion, an elixir, a gel, an ointment, a cream, or a suppository. In another embodiment, the composition is in the form of a capsule.

In one embodiment, the composition is in a form suitable for intracranial administration. In another embodiment, the composition is in a form suitable for intranasal administration. In another embodiment, the composition is in a form suitable for oral, intravenous, intraarterial, intramuscular, subcutaneous, parenteral, transmucosal, transdermal, or topical administration.

In one embodiment, the composition is a controlled release composition. In another embodiment, the composition is an immediate release composition. In another embodiment, the composition is a liquid dosage form. In another embodiment, the composition is a solid dosage form.

In one embodiment, this invention provides a method for inducing or enhancing neurogenesis in a subject, said method comprising administering to a subject a composition comprising:
a) an agent which increases brain levels of interferon-γ; and
b) an agent which reduces the number of T regulatory (Treg) cells.

In another embodiment, the method further comprises a method of administering an agent which suppresses neurotoxic inflammatory brain responses.

In one embodiment, the method comprises a method of administering to a subject a composition formulated for brain-specific delivery. In another embodiment, the method comprises administering a composition comprising a liposome.

In one embodiment, the method comprises administering a composition to a subject wherein said subject is afflicted with a neurodegenerative disease or disorder. In one embodiment, the neurodegenerative disease or disorder comprises an injury, disease, disorder or condition of the central nervous system (CNS). In another embodiment, the neurodegenerative disease or disorder comprises Alzheimer's disease, Parkinson's disease, Huntington's disease, multiple sclerosis, diabetic neuropathy or amyotrophic lateral sclerosis (ALS). In another embodiment, the neurodegenerative disease or disorder comprises spinal cord injury, closed head injury, blunt trauma, penetrating trauma, hemorrhagic stroke, ischemic stroke, cerebral ischemia, optic nerve injury, or injury caused by tumor excision. In another embodiment, the subject is at risk for a neurodegenerative disease or disorder.

In one embodiment, the method comprises administering a composition to a subject in a form suitable for administration via an intracranial route. In another embodiment, the composition as set forth herein is in a form suitable for administration via an intranasal route. In another embodiment, the method as set forth herein comprises a method of administering a composition as set forth herein via an oral, intravenous, intraarterial, intramuscular, subcutaneous, parenteral, transmucosal, transdermal, or topical route.

In one embodiment, the method further comprises administering neurotrophins. In another embodiment, said neurotrophins comprise BDNF, NT-3 or NT4.

In one embodiment, this invention provides a kit for inducing or enhancing neurogenesis in a subject, said kit comprising:
a) an agent which increases brain levels of interferon-γ; and
b) an agent which reduces the number of T regulatory (Treg) cells.

In another embodiment, the kit further comprises an agent which suppresses neurotoxic inflammatory brain responses.

In one embodiment, the kit comprises a liposome.

In one embodiment, the kit comprises agents formulated for administration to a subject wherein said subject is afflicted with a neurodegenerative disease or disorder. In one embodiment, the neurodegenerative disease or disorder comprises an injury, disease, disorder or condition of the central nervous system (CNS). In another embodiment, the neurodegenerative disease or disorder comprises Alzheimer's disease, Parkinson's disease, Huntington's disease, multiple sclerosis, diabetic neuropathy or amyotrophic lateral sclerosis (ALS). In another embodiment, the neurodegenerative disease or disorder comprises spinal cord injury, closed head injury, blunt trauma, penetrating trauma, hemorrhagic stroke, ischemic stroke, cerebral ischemia, optic nerve injury, or injury caused by tumor excision. In another embodiment, the subject is at risk for a neurodegenerative disease or disorder.

In one embodiment, the kit comprises agents formulated for administration to a subject via an intracranial route. In another embodiment, the kit comprises agents formulated for administration to a subject via an intranasal route. In one embodiment, the kit comprises agents formulated for administration to a subject via an oral, intravenous, intraarterial, intramuscular, subcutaneous, parenteral, transmucosal, transdermal, or topical route.

In one embodiment, the kit further comprises neurotrophins. In another embodiment, said neurotrophins comprise BDNF, NT-3 or NT4.

### BRIEF DESCRIPTION OF THE FIGURES

FIG. 1. Quantitative PCR analysis of IFN-γ in the brains of IFN-γ Tg mice. A. Expression of IFN-γ mRNA in the brains of IFN-γ Tg mice relative to wild-type controls at one, three, and nine months of age. B. Determination of the amounts of IFN-γ protein in the brains of IFN-γ Tg mice. The correlation between IFN-γ mRNA (fold-change) and IFN-γ protein detected by ELISA in each concentration of anti-CD3 is shown.

FIG. 2. Neurogenesis is increased in adult and old IFN-γ Tg mice. Amounts of newly generated neuronal precursor cells in the DG were measured in the brains of mice injected with BrdU for eight days (A-D) or three days (E). A. Two-dimensional projection of Z-stack images taken in the DG demonstrating single-positive DCX (red) and BrdU (green) cells, and 3-D projection image demonstrating the double-positive cells (right panel). B. BrdU/DCX immunostaining of sagittal brain sections from three-month-old and nine-month-old wild-type (left panels) and IFN-γ Tg (right panels) mice. C. Quantitative analysis of BrdU and BrdU/DCX double-positive cells in sagittal brain sections from three-month-old wild-type and IFN-γ Tg mice. D. Quantitative analysis of BrdU, DCX, and BrdU/DCX double-positive cells in sagittal brain sections from nine-month-old wild-type and IFN-γ Tg mice. E. Quantitative analysis of BrdU and BrdU/DCX double-positive cells in sagittal brain sections from one-month-old (left panel) and three-month-old (right panel) wild-type and IFN-γ Tg mice injected three times with BrdU. P values and SEM were calculated from a representative experiment with five mice in each group using the two-tailed Mann-Whitney test.

FIG. 3. Neurogenesis is increased in old APP/IFN-γ double Tg mice. A. Aβ plaques (blue), CD11b microglia (green), and their merge image (right panel) in the hippocampus of 9-month-old APP Tg mice. B. Immunostaining of BrdU (green) and DCX (red) cells in the DG of nine-month-old wild-type, APP, and APP/IFN-γ Tg mice. Nuclei were counterstained with TOPRO-3 (blue), a stain specific for nucleic acids. C. Stereological quantification analysis of BrdU, DCX, and BrdU/DCX double-positive cells in sagittal brain sections. D. Average neurogenesis rate in one-, three-, and nine-month-old wild-type and nine-month-old APP Tg mice (white columns), where one-month-old mice represent 100%. The percent increase in neurogenesis in age-matched IFN-γ Tg mice is shown in the black columns. P values and SEM were calculated from a representative experiment with five mice in each group using the two-tailed Mann-Whitney test.

FIG. 4. Oligodendrogenesis is increased in APP Tg but not in IFN-γ Tg mice. Sagittal brain sections from nine-month-old wild-type, APP, IFN-γ, and APP/IFN-γ Tg mice were immunolabeled with anti-NG2 (green) (oligodendrocyte progenitor cell-marker), anti-BrdU (red), and TO-PRO-3 for nuclear counterstaining (blue). A. The DG-hippocampus area of wild-type mice demonstrating the distribution of BrdU/NG2 double-positive cells. B. NG2/CD11b immunostaining demonstrating single-positive NG2 (red) and CD11b (green) cells in the hippocampus of wild-type mice. C. Three-dimensional projection of Z-stack images demonstrating BrdU/NG2 double-positive cells. D. Stereological quantitative analysis of BrdU/NG2 double-positive cells in the hippocampi of wild-type, APP, IFN-γ, and APP/IFN-γ Tg mice. P values and SEM were calculated from a representative experiment with four mice in each group using the two-tailed Mann-Whitney test.

FIG. 5. Synaptophysin immunoreactivity is increased in the hippocampus of IFN-γ Tg mice. Synaptophysin immunoreactivity (green) was measured in the CA1 and CA3 hippocampus regions and the molecular layer of the DG of nine-month-old wild-type, APP, IFN-γ, and APP/IFN-γ Tg mice. A. Low magnification image demonstrating the areas in the hippocampus analyzed for quantification. B. Higher magnification of representative images taken of the CA1 area of wild-type, APP, and APP/IFN-γ Tg mice. C. Quantitative stereological analysis of the percentage fluorescent area of each region performed on three randomized images obtained from three sections per mouse. P values and SEM were calculated from at least four mice in each group using the two-tailed Mann-Whitney test.

FIG. 6. IFN-γ Tg mice perform better in the spatial learning and memory cognitive test. A. In the acquisition phase, there were no significant differences between the groups in their abilities to find the hidden platform throughout the five-day experiment. B. A probe-trial test (indicative of memory) performed on day 6, i.e., one day after the last training day. The graph represents the percentage of time (in 60 s) the mouse spent in the quadrant that had previously contained the platform. C-E. The reversal phase of the task where three platform locations were used successively, one at a time for two days. The IFN-γ Tg mice took significantly less time than the controls to find the hidden platform on days 9 and 11.

FIG. 7. Accumulation of amyloid beta in the brain. A-C: Brains were labeled with an antibody against Aβ (blue) and imaged by a confocal microscope. Accumulation of Aβ plaques is more abundant and intense as the mice become older (9-, 16-month-old). Sections taken from young mice do not exhibit plaque formation (no staining in 3-month-old mice).

FIG. 8. A-C: Brains were labeled with an antibody against CD11b (green) and imaged by a confocal microscope. Immunoreactivity of CD11b is observed mainly around the plaques (intense green) and become more intense as the mice become older (9-, 16-month-old). Sections taken from young mice do not exhibit CD11b staining. D: Ramified microglia (green) surrounding the Aβ plaque (blue). Virtual Z-stack image taken from section of 30 μm by a confocal microscope (×60).

FIG. 9. Astrocyte activation. A-D: brains were labeled with an antibody against GFAP (blue). WT and APP Tg mice in 9-month-old mice show more pronounced GFAP immunoreativity.

FIG. 10. Neurogenesis is decreased in DG of aged mice. A,B: Images show the DG area of 3-month-old (A) and 9-month-old (B) WT mice. C,D: Quantification of proliferating cells (C) and proliferating neurons (D) in the DG area of 3- and 9-month-old WT mice.

FIG. 11. Neurogenesis is decreased in the DG of APP Tg mice. The graph shows the average number of BrdU+ cells in the DG per brain section of 30 μm in 3- and 9-month-old WT and APP Tg mice. A significant difference in proliferative cells is observed in 9-month-old APP Tg mice.

FIG. 12. Effect of IFN-γ on neurogenesis. A: Neurogenesis in 3- and 9-month-old WT and IFN-γ Tg mice. The graph shows average number of BrdU+ cells in the DG per brain section of 30 μm. Significant elevation in proliferative cells is observed in 9-month-old IFN-γ Tg mice. B: Neurogenesis in 3- and 9-month-old APP and APP/IFN-γ Tg mice. A significant difference of up to 2-fold increase of BrdU+ cells is observed in APP/IFN-γ compared to APP Tg mice.

FIG. 13. Effect of IFN-γ on oligodendrogenesis. A-D: Brains were labeled with anti-NG2 antibody (green), anti-BrdU antibody (red) and TO-PRO3 which stains cellular DNA. Images of the DG area were taken using confocal microscopy. Images on left present an overview of the observed area, while images on the right show high magnification of the highlighted box in the left image. Arrows indicate cells double-labeled cells with NG2/BrdU. E: Quantification of NG2/BrdU+ cells was done in at least two animals and at least three tissues from each mouse. Cells surrounding the DG area were counted (granule and subgranular zone) and their average number was calculated.

FIG. 14. Aβ immunization results in immune cell infiltration into the CNS. Ten-month-old APP/IFN-γ mice were immunized with Aβ and killed 19 days later. (a) CD11b resident microglia and infiltrating monocytes/MF (green). (b) CD4 T cells (green). (c) CD8 T cells (green). (d) CD19 B cells (green). Red arrows indicate infiltrates in parenchymal vessels. Yellow arrowheads indicate meningeal infiltrates. Counter-staining was performed with TOPRO-3 (blue). Bars represent 200 mm in (a) and 100 mm in (b), (c) and (d).

FIG. 15. T cells cross into the paremchyma and migrate to Aβ plaques. Mice were treated as described in FIG. 7. Tissue stained for CD4 (red), CD8 (green) and counter-stained with TOPRO-3 (blue) in (a) or stained for Aβ (blue) in (b). High magnification in (c) and (d) showing amyloid plaques targeted by both CD4 and CD8 T cells. (e) Section was stained for CD4 (red), CD11b (green) and Aβ (blue). (f) 3D reconstitution of the image in (e) using Volocity of software. Bars represent 100 mm in (a), (b), and 20 mm in (c), (d), (e).

FIG. 16. Up-regulation of adhesion molecules following Aβ vaccination of APP/IFN-γ Tg mice. Ten-month-old APP/IFN-γ Tg mice were immunized with Aβ/CFA and killed 19 days later, or left untreated. (a) Brain sections were stained for ICAM-1 (red) and PECAM-1 (green) and were counter-stained with TOPRO-3 (blue). (b) Brain sections were stained for VCAM-1 (red) and PECAM-1 (green) and were counter-stained with TOPRO-3 (blue). Arrows represent areas of high magnification. (c) Quantitative analysis of ICAM-1 and VCAM-1 on parenchymal and meningeal blood vessels in the brain. Bars for upper and middle panel represent 100 mm, lower panel represent 20 mm.

FIG. 17. CD11c+dendritic cells localize to the perivascular area following Aβ vaccination. Following Aβ vaccination of APP/IFN-γ mice, CD11c⁺ dendritic cells localize to the perivascular area and are in contact with CD4 T cells. APP/IFN-γ Tg mice were vaccinated with Aβ/CFA and were sacrificed after 13 (a) or 19 days (c). Brain tissues were collected and stained for PECAM-1 (green), CD11c (red) and counter-stained with TOPRO-3 in (a,b) or stained for CD4 in (c, d, e, f) (blue). High magnification images show CD11c⁺ dendritic cells appearing in the perivascular zone, on the outer edges of blood vessels and in contact with infiltrating CD4 T cells.

FIG. 18. Effect of PLP vaccination in APP/IFN-γ transgenic mice. Nine-month-old APP/IFN-γ were vaccinated with PLP/CFA and killed 19 days later. (a) Brain section showing CD4 T-cells (green) in the meninges, but not in the parenchyma. Counter-stained with TOPRO-3 (blue). (b), (c) CD11c dendritic cells (red) localized to blood vessels (green) in the parenchyma and (d) in the meninges, where they are in contact with meningeal CD4 T cells (blue). Scale represents 100 mm in (a) and 20 mm in (b), (c) and (d).

FIG. 19. Aβ immunization results in BDNF secretion by glia cells. (a) BDNF (green) staining in untreated APP/IFN-γ mouse. (b) BDNF (green) and CD4 (red) staining in Aβ-immunized mouse. White arrows indicate sample glia cells expressing BDNF. (c) and (d) BDNF (green) and CD4 (red) staining of magnified areas from immunized mice showing CD4 T cell-BDNF-expressing glia cell interaction, marked with white arrow heads. (e) BDNF (green) and (f) NewN (red) staining in untreated mice. Merged image in (g). (h, k) BDNF (green) expressing (i, 1) GFAP (red) astrocytes. Merged images in (j) and (m). TOPRO-3 counter-staining shown in blue. Bars represent 100 mm in (a, b), 20 mm in all other images.

FIG. 20. Prevalence of CD4+CD25+ and CD4+FOXP3+ T cells in aged IFN-γ Tg mice. Spleen cells from B6 \SJL, B6 \INF-γ, APP and APP/IFN-γ mice were stained with FITC anti-CD4, PE anti-CD25 and APC anti-FOXP3 Ab and analyzed by flow cytometry. Results shown are representative of two independent experiments.

FIG. 21. CD4+CD25+ Treg numbers are influenced by aging, AD and IFN-γ. Spleen cells from B6\SJL, B6\INF-γ, APP and APP/IFN-γ mice were stained with FITC anti-CD4, PE anti-CD25 and APC anti-FOXP3 and analyzed by flow cytometry.

FIG. 22. The effect of Treg depletion on learning and memory. A,B. In the acquisition phase, there were no significant differences between Treg-depleted wild-type and wild-type mice. C. Significant differences in acquisition rates between young and old (24-month-old) IFN-γ Tg mice were seen. D. Depletion of Tregs in IFN-γ Tg mice improves their cognitive function as compared to non-Treg-depleted IFN-γ Tg mice.

### DETAILED DESCRIPTION OF THE INVENTION

This invention provides, in some embodiments, a method for treating a subject suffering from a disease or disorder of the nervous system, associated with an inflammatory response. This invention further provides a pharmaceutical composition for treating a subject suffering from a disease of the central nervous system, including, inter alia, an agent which increases brain levels of interferon-γ; and/or an agent which reduces the number of brain T regulatory (Treg) cells, and/or further comprising an agent, which suppresses neurotoxic inflammatory brain responses.

In one embodiment, the term “neurogenesis” refers to the formation of new neurons. In some embodiments, the term “neurogenesis” refers to the differentiation of stem or progenitor cells into neurons. In some embodiments, “neurogenesis” may occur in the central nervous system (CNS), or in some embodiments, in the peripheral nervous system (PNS). In some embodiments, “neurogenesis” may occur in the neurogenic niches of the subventricular zone of the lateral ventricles, or in some embodiments, “neurogenesis” may occur and the subgranular zone of the hippocampal dentate gyrus. In another embodiment, neurogenesis can also be induced in non-neurogenic brain areas. In some embodiments, neurogenesis is promoted by adaptive immune responses, and negatively impacted by inflammatory, non-specific responses in the nervous system.

In some embodiments, neurogenesis may be determined by changes in surface marker expression. In some embodiments, such markers may comprise, inter alia, doublecortin, polysialylated nerve cell adhesion molecule, neurogenic differentiation factor, TUC-4, or combinations thereof, or others.

In one embodiment, the compositions of the invention comprise an agent which increases brain levels of interferon-γ.

In one embodiment, the term “agent” refers to any molecule which satisfies the indicated purpose. In some embodiments, “agent” is a nucleic acid, an oligonucleotide, an oligopeptide, a polypeptide, a protein, a functional fragment thereof, a small molecule, or any chemical moiety suitable for the indicated purpose.

In one embodiment, the agent is a nucleic acid. In some embodiments, the term “nucleic acid” refers to polynucleotide or to oligonucleotides such as deoxyribonucleic acid (DNA), and, where appropriate, ribonucleic acid (RNA) or mimetic thereof. The term should also be understood to include, as equivalents, analogs of either RNA or DNA made from nucleotide analogs, and, as applicable to the embodiment being described, single (sense or antisense) and double-stranded polynucleotides. This term includes oligonucleotides composed of naturally occurring nucleobases, sugars and covalent internucleoside (backbone) linkages as well as oligonucleotides having non-naturally-occurring portions, which function similarly. Such modified or substituted oligonucleotides are often preferred over native forms because of desirable properties such as, for example, enhanced cellular uptake, enhanced affinity for nucleic acid target and increased stability in the presence of nucleases.

As will be appreciated by one skilled in the art, a fragment or derivative of a nucleic acid sequence or gene that encodes for a protein or peptide can still function in the same manner as the entire, wild type gene or sequence. Likewise, forms of nucleic acid sequences can have variations as compared to wild-type sequences, nevertheless encoding the protein or peptide of interest, or fragments thereof, retaining wild-type function exhibiting the same biological effect, despite these variations. Each of these represents a separate embodiment of this present invention.

The nucleic acids of this invention can be produced by any synthetic or recombinant process such as is well known in the art. Nucleic acids can further be modified to alter biophysical or biological properties by means of techniques known in the art. For example, the nucleic acid can be modified to increase its stability against nucleases (e.g., “end-capping”), or to modify its lipophilicity, solubility, or binding affinity to complementary sequences.

DNA according to the invention can also be chemically synthesized by methods known in the art. For example, the DNA can be synthesized chemically from the four nucleotides in whole or in part by methods known in the art. DNA can also be synthesized by preparing overlapping double-stranded oligonucleotides, filling in the gaps, and ligating the ends together, by standard methods known in the art. DNA expressing functional homologues of the protein can be prepared from wild-type DNA by site-directed mutagenesis. The DNA obtained can be amplified by methods known in the art. One suitable method is the polymerase chain reaction (PCR) method. Such methods are well known in the art, see for example, U.S. Pat. No. 4,683,195, “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are well known in the art and are provided for the convenience of the reader. All the information contained in any reference cited herein is to be understood to be incorporated by reference in its entirety.

Methods for modifying nucleic acids to achieve specific purposes are disclosed in the art, for example, in Sambrook et al. (1989). Moreover, the nucleic acid sequences of the invention can include one or more portions of nucleotide sequence that are non-coding for the protein of interest. Variations in the DNA sequences, which are caused by point mutations or by induced modifications (including insertion, deletion, and substitution) to enhance the activity, half-life or production of the polypeptides encoded thereby, are also encompassed in the invention.

In some embodiments, the agent, which increases brain levels of interferon-γ is a nucleic acid encoding the protein, or a functional fragment thereof. In some embodiments, the agent is the protein or a functional polypeptide fragment thereof. In some embodiments, the term “functional fragment” refers to the ability of the fragment to effect the indicated purpose of the cited agent. According to this aspect, and in some embodiments, functional fragments of agents, which increase brain levels of interferon-γ, may comprise a fragment of the polypeptide, which may still effect neurogenesis as described herein.

In some embodiments, the nucleic acid agent, which increases brain levels of interferon-γ, encodes the protein. In some embodiments, the nucleic acid may have a sequence corresponding to or homologous to any known sequence encoding the protein, or one inducing the same. For example, and in some embodiments, the nucleic acid may have a sequence corresponding to or homologous to that set forth in NCBI's Genbank Accession No.: NM000619, EF173872, E12009, E11745, E15793, E15653, E06017, E00832, E00692, E00663, E00611, E00388, E00380, E00270, E00228, E00226, E00180, E00119, E00118, and others. In some embodiments, the nucleic acid agent, which increases brain levels of interferon-γ encodes interleukin-2 (IL-2), IL-12, IL-18, interferon α, interferon β or TNF α, comprising any sequence known to encode the same, for example, the nucleic acid may have a sequence corresponding to or homologous to that set forth in NCBI's Genbank Accession No.: NM_{—}000594, NM_{—}000882, NM_{—}002176, or others, as will be appreciated by one skilled in the art.

In one embodiment, the terms “homology”, “homologue” or “homologous”, in any instance, indicate that the sequence referred to, whether an amino acid sequence, or a nucleic acid sequence, exhibits at least 70% correspondence with the indicated sequence. In another embodiment, the amino acid sequence or nucleic acid sequence exhibits at least 72% correspondence with the indicated sequence. In another embodiment, the amino acid sequence or nucleic acid sequence exhibits at least 75% correspondence with the indicated sequence. In another embodiment, the amino acid sequence or nucleic acid sequence exhibits at least 77% correspondence with the indicated sequence. In another embodiment, the amino acid sequence or nucleic acid sequence exhibits at least 80% correspondence with the indicated sequence. In another embodiment, the amino acid sequence or nucleic acid sequence exhibits at least 82% correspondence with the indicated sequence. In another embodiment, the amino acid sequence or nucleic acid sequence exhibits at least 85% correspondence with the indicated sequence. In another embodiment, the amino acid sequence or nucleic acid sequence exhibits at least 87% correspondence with the indicated sequence. In another embodiment, the amino acid sequence or nucleic acid sequence exhibits at least 90% correspondence with the indicated sequence. In another embodiment, the amino acid sequence or nucleic acid sequence exhibits at least 92% correspondence with the indicated sequence. In another embodiment, the amino acid sequence or nucleic acid sequence exhibits at least 95% or more correspondence with the indicated sequence. In another embodiment, the amino acid sequence or nucleic acid sequence exhibits 95%-100% correspondence to the indicated sequence. Similarly, as used herein, the reference to a correspondence to a particular sequence includes both direct correspondence, as well as homology to that sequence as herein defined.

In one embodiment, the term “homology”, when in reference to any nucleic acid sequence indicates a percentage of nucleotides in a candidate sequence that are identical with the nucleotides of a corresponding native nucleic acid sequence.

Homology may be determined in the latter case by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

An additional means of determining homology is via determination of candidate sequence hybridization, methods of which are well described in the art (See, for example, “Nucleic Acid Hybridization” Hames, B. D., and Higgins S. J., Eds. (1985); Sambrook et al., 1989, Molecular Cloning, A Laboratory Manual, (Volumes 1-3) Cold Spring Harbor Press, N.Y.; and Ausubel et al., 1989, Current Protocols in Molecular Biology, Green Publishing Associates and Wiley Interscience, N.Y). For example, methods of hybridization may be carried out under moderate to stringent conditions, to the complement of a DNA encoding a native caspase peptide. Hybridization conditions being, for example, overnight incubation at 42° C. in a solution comprising: 10-20% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared salmon sperm DNA.

In some embodiments, the agent is a vector comprising a nucleic acid as described herein. In some embodiments, vectors are generated as follows: polynucleotides encoding sequences of interest can be ligated into commercially available expression vector systems suitable for transducing/transforming eukaryotic and for directing the expression of recombinant products within the transduced/transformed cells. It will be appreciated that such commercially available vector systems can easily be modified via commonly used recombinant techniques in order to replace, duplicate or mutate existing promoter or enhancer sequences and/or introduce any additional polynucleotide sequences such as for example, sequences encoding additional selection markers or sequences encoding reporter genes.

According to another embodiment, nucleic acid vectors comprising the isolated nucleic acid sequences encoding for the protein of interest include a regulatory element, such as a promoter for regulating expression of the isolated nucleic acid. Such promoters are known to be cis-acting sequence elements required for transcription as they serve to bind DNA dependent RNA polymerase, which transcribes sequences present downstream thereof.

The vector may, in another embodiment, comprise an inducible promoter, or one that expresses the sequences of interest constitutively.

Nucleotide sequences which regulate expression of a gene product, which are in one embodiment, promoter and enhancer sequences, are selected, in another embodiment, based upon the type of cell in which the gene product is to be expressed, or in another embodiment, upon the desired level of expression of the gene product, in cells infected with the vectors of the invention. According to this aspect of the invention, the gene product corresponds to the heterologous protein, as described herein. Regulated expression of such a heterologous protein may thus be accomplished, in one embodiment.

For example, a promoter known to confer cell-type specific expression of a gene linked to the promoter can be used. A promoter specific for various neural cell-specific regulatory elements, including neural dystrophin, neural enolase and A4 amyloid promoters are used, in some embodiments. In another embodiment, a regulatory element, which can direct constitutive expression of a gene in a variety of different cell types, such as a viral regulatory element, can be used. Examples of viral promoters commonly used to drive gene expression include those derived from polyoma virus, adenovirus 2, cytomegalovirus and Simian Virus 40, and retroviral LTRs.

In another embodiment, a regulatory element, which provides inducible expression of a gene linked thereto can be used. The use of an inducible regulatory element (e.g., an inducible promoter) allows for modulation of the production of the gene product in the cell. In another embodiment, the inducible regulatory systems for use in eukaryotic cells include hormone-regulated elements (e.g., see Mader, S, and White, J. H. (1993) Proc. Natl. Acad. Sci. USA 90:5603-5607), synthetic ligand-regulated elements (see, e.g., Spencer, D. M. et al. 1993) Science 262:1019-1024) and ionizing radiation-regulated elements (e.g., see Manome, Y. Et al. (1993) Biochemistry 32:10607-10613; Datta, R. et al. (1992) Proc. Natl. Acad. Sci. USA 89:1014-10153). Additional tissue-specific or inducible regulatory systems may be developed for use in accordance with the invention.

A vector according to the present invention, may, in another embodiment further include an appropriate selectable marker. The vector may further include an origin of replication, and may be a shuttle vector, which can propagate both in prokaryotic, and in eukaryotic cells, or the vector may be constructed to facilitate its integration within the genome of an organism of choice. The vector, in other embodiments may be, for example, a plasmid, a bacmid, a phagemid, a cosmid, a phage, a virus or an artificial chromosome. In another embodiment, the vector is a viral particle comprising the nucleic acids of the present invention. In another embodiment, this invention provides liposomes comprising the nucleic acids and vectors of this invention. Methods for preparing such liposomes are well known in the art, and may be as described in, for example WO 96/18372; WO 93/24640; Mannino and Gould-Fogerite (1988) BioTechniques 6(7): 682-691; Rose U.S. Pat. No. 5,279,833; WO 91/06309; and Feigner et al. (1987) Proc. Natl. Acad. Sci. USA 84: 7413-7414).

In some embodiments, the nucleic acid agent, which increases brain levels of interferon-γ is the protein itself or a functional fragment or derivative thereof. In some embodiments, the polypeptide may have a sequence corresponding to or homologous to any known sequence for the protein, or one inducing the same. For example, and in some embodiments, the polypeptide may have an amino acid sequence corresponding to or homologous to that set forth in NCBI's Genbank Accession No.: AAM28885, CAA44325, CAβ17327, AAK95388, ABM53145, AAβ20100, AAβ20098, AAK53058, CAA00226, AAA53230, AAA16521, CAA44328, CAA44329, CAA44330, CAA44326, and others. In some embodiments, the polypeptide agent, which increases brain levels of interferon-γ is interleukin-2 (IL-2), IL-12, IL-18, interferon α, interferon β or TNF α, comprising any sequence known corresponding to the same, for example, the polypeptide may have an amino acid sequence corresponding to or homologous to that set forth in NCBI's Genbank Accession No.: AAA52716, AAA52724, AAA52713, AAC41702, AAO26357, NP_{—}002167, CAA01199, AAA59140, AAA98792, AAD16432, ABM53138, NP_{—}001553, AAK95950, CAC01436 or others, as will be appreciated by one skilled in the art.

In another embodiment, the “agent” encodes a piggyback molecule, enabling its entry into the brain, for example a brain specific interacting protein such as an antibody or a fragment thereof interacting specifically with brain-specific proteins.

In some embodiments, the agent increases brain levels of interferon-γ. In some embodiments, the phrase “increases brain levels of interferon-γ” refers to a brain-exclusive phenomenon, such that brain levels are exclusively enhanced, or in some embodiments, preferentially enhanced, or in some embodiments, brain levels are enhanced, while peripheral levels are enhanced as well. In some embodiments, the phrase “increases brain levels of interferon-γ” refers to increased production of the interferon in the brain, or in some embodiments, increased production predominantly in the brain, or in some embodiments, increased production peripherally, which enables increased presence in the brain.

In one embodiment, the agent is interleukin-2 (IL-2). In another embodiment, the agent is IL-12. In another embodiment, the agent is IL-18. In another embodiment, the agent is interferon α or interferon β or tumor necrosis factor α. In another embodiment, the agent is lymphocyte growth hormone.

In another embodiment, the agent is derived from Brassica vegetables. In another embodiment, the agent is 3,3′-di-indolylmethane (DIM). In another embodiment, the agent is indole-3-carbimol.

In another embodiment, the agent is any compound, composition or agent which initiates a cell-mediated immune response in the brain.

In another embodiment, the agent is any compound, composition or agent which initiates a cell-mediated immune response in the central nervous system (CNS), which comprises the brain and the spinal cord. In another embodiment, the agent is any compound, composition or agent which initiates a cell-mediated immune response in the peripheral nervous system (PNS). In another embodiment, the agent is any compound, composition or agent which initiates a cell-mediated immune response in both the CNS and the PNS.

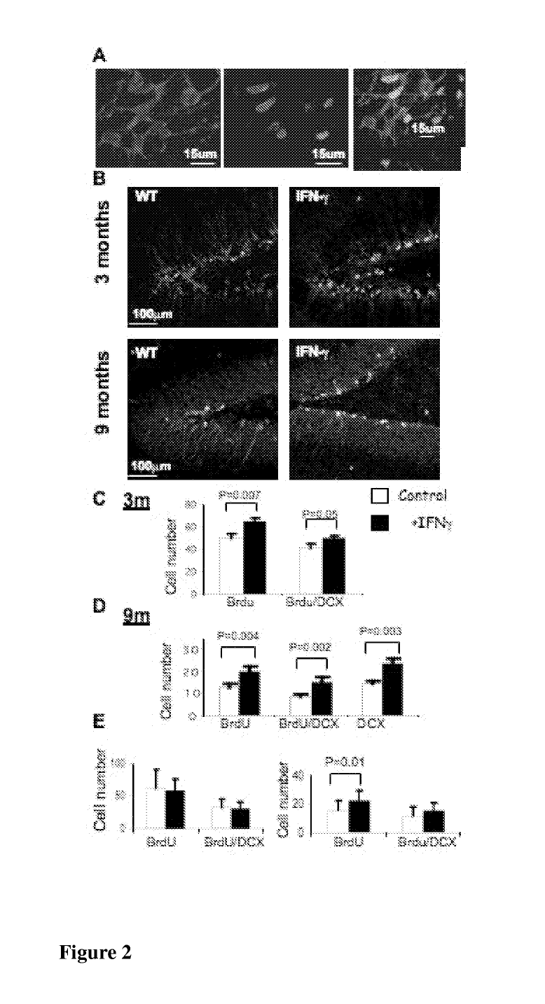

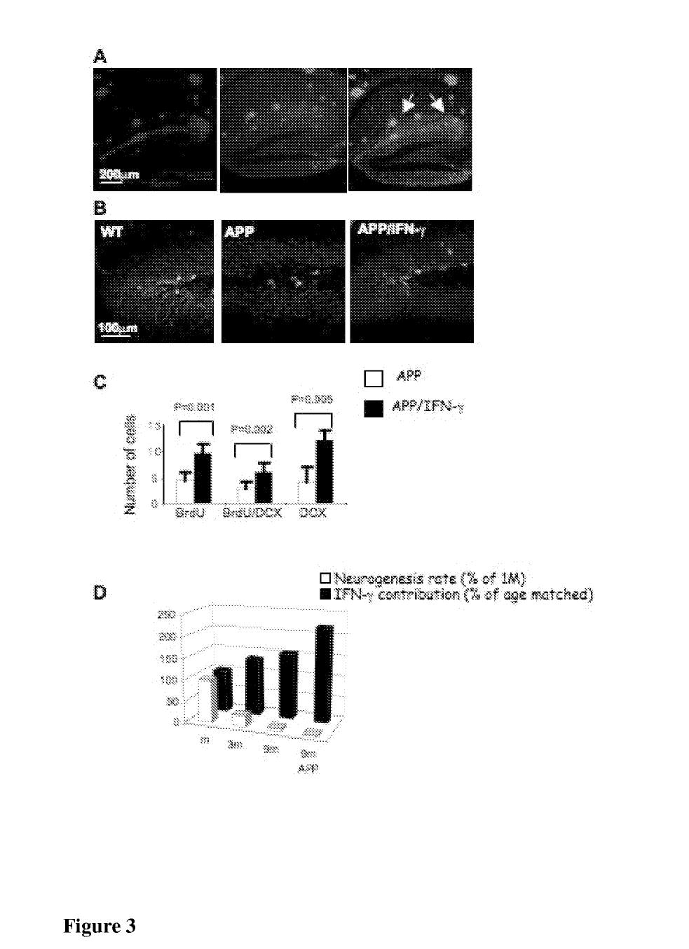

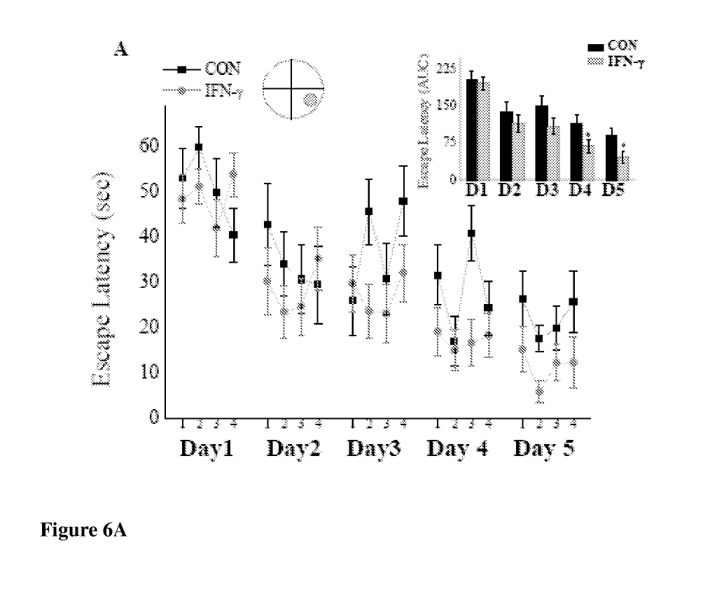

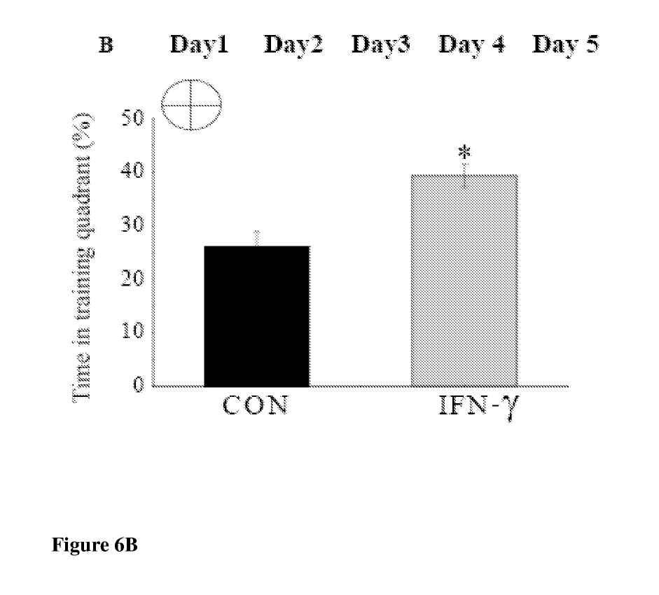

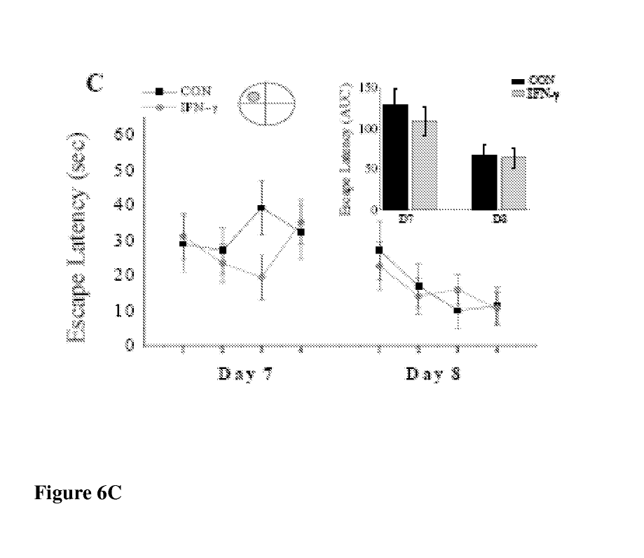

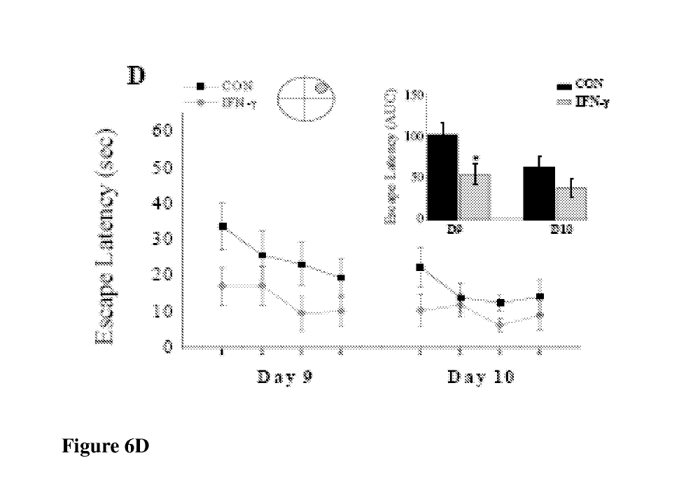

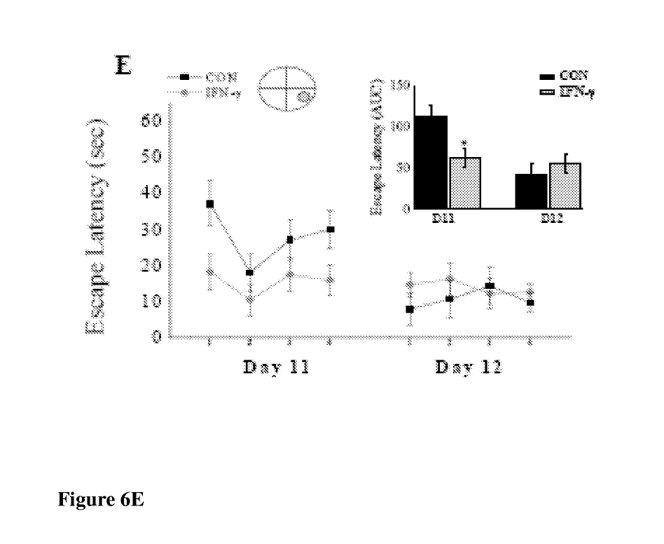

IFN-γ was shown herein to significantly increase the proliferation of NPCs in the DG and their differentiation to the neuronal lineage in adult mice concomitant with improved cognitive performance. This effect of IFN-γ on neurogenesis was greater in the DG of aged mice and APP Tg mice where Aβ accumulated extensively throughout the course of AD. The low amounts of IFN-γ in the brain induced a mild up-regulation of cytokines and neurotrophic factors known to regulate the fate of NPCs at the DG (FIGS. 2 and 3). Three-month-old IFN-γ Tg mice also had better spatial learning and memory performances compared with control wild-type mice (FIG. 6), suggesting that cognitive functions declines at this age and can be improved with enhanced neurogenesis.

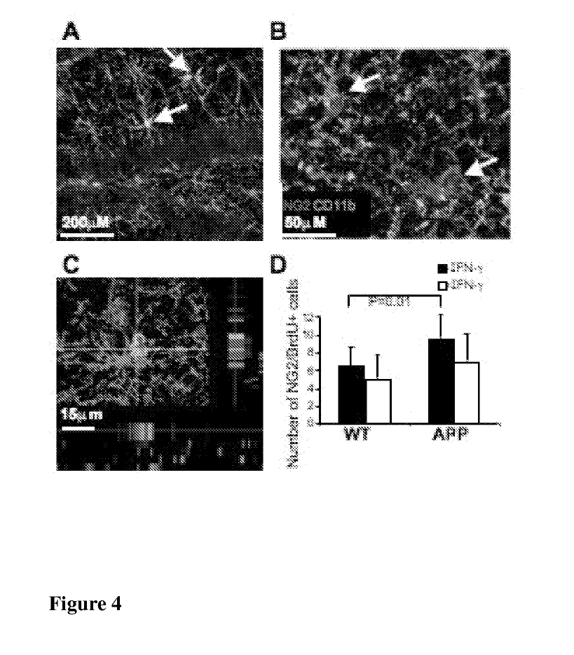

IFN-γ was also shown to induce neuronal precursor cell proliferation and differentiation already in the adult DG concomitant with age-related decline in neurogenesis. In accordance with the enhanced neurogenesis observed, IFN-γ reduced oligodendrogenesis, which was otherwise increased in the hippocampus of APP Tg mice (FIG. 4).

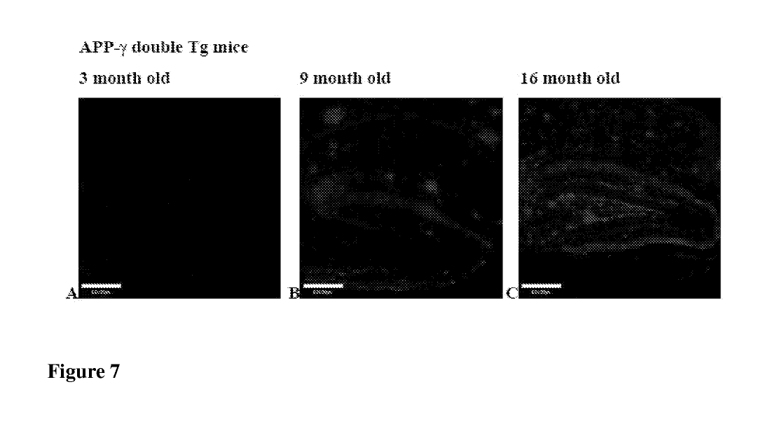

Amyloid accumulation in the brain causes further decline in DG neurogenesis compared with normal aging of the mice. As in wild-type mice, the decline in neurogenesis at 9 months of age was milder in APP/IFN-γ compared with APP-Tg mice (FIG. 7).

In one embodiment, the composition of the invention comprises an agent, which reduces the number of brain T regulatory (Treg) cells.

T regulatory cells (Tregs) are a regulatory component of the immune system and act to modulate immune responses.

In some embodiments, the term “Tregs”, refers to a T cell population that inhibits or prevents the activation, or in another embodiment, the effector function, of another T lymphocyte. In one embodiment, the Tregs are a homogenous population, or in another embodiment, a heterogeneous population.

The Tregs of this invention express CD25 and CD4 on their cell surface. In one embodiment, the Tregs may be classified as CD25^{high} expressors, or in another embodiment, the Tregs may be classified as CD4^{low} expressors, or in another embodiment, a combination thereof. In another embodiment, the Tregs may express CTLA-4, or in another embodiment, GITR. In one embodiment, the Tregs may be classified as CTLA-4^{high} expressors, or in another embodiment, the Tregs may be classified as GITR^{high} or in another embodiment, a combination thereof. In another embodiment, the Tregs of this invention are CD69-. In another embodiment, the Tregs of this invention are CD62L^{hi}, CD45RB^{lo}, CD45RO^{hi}, CD45RA-, αE137 integrin, Foxp3, expressors, or any combination thereof. It is to be understood that the agent, which reduces the number of brain Tregs of this invention encompasses Treg cell populations expressing any number or combination of cell surface markers, as described herein, and as is well known in the art, and are to be considered as part of this invention.

In some embodiments, the phrase “T regulatory cells (Tregs)” refers to any cell population with such activity, for example, as described in U.S. Pat. No. 7,115,259, US Patent Application Publication No. US2005000244142, and US Patent Application Publication No. US20070172947A1.

In one embodiment, Tregs have unique cell surface expression profiles. In one embodiment, Tregs express CD4 or CD8, CD25 and Foxp3. In one embodiment, Tregs mediate their suppressive activity via the production of inhibitory cytokines, which, in one embodiment, is IL-10, and in another embodiment, is tumor growth factor-β (TGF-β).

In one embodiment, the agent reduces the number of brain T regulatory (Treg) cells) by a peripheral route. In another embodiment, the agent reduces the number of brain Treg cells by a local route. In one embodiment, the agent reduces the number of brain T regulatory (Treg) cells) by a direct route. In another embodiment, the agent reduces the number of brain Treg cells by an indirect route. In one embodiment, the reduction in the number of brain Treg cells is via a mechanism ameliorating or abrogating Treg education. In another embodiment, reduction in the number of brain Treg cells is via Treg cells lysis. In another embodiment, the reduction in the number of brain Treg cells is a function of anergy of the Treg cells. In one embodiment, IFN-γ participates in any or all of the above phenomenon resulting in a reduction in the number of brain Treg cells.

In one embodiment, the agent is brain-specific. In another embodiment, the agent has an affinity for brain-derived peptides. In one embodiment, the agent is a neutralizing antibody. In another embodiment, the agent may be an antibody, which, in one embodiment, is in an inactive form until it reaches the brain, and is then cleaved by a brain-specific enzyme to an active form. In another embodiment, the agent is specific for Treg cells. In another embodiment, the agent prevents CD25 expression on precursor cells indirectly. In one embodiment, the agent is a peptide. In another embodiment, the agent is a protein. In another embodiment, the agent is a small molecule. In another embodiment, the agent is antigen-specific. In another embodiment, the agent has an affinity for self peptides.

In one embodiment, the agent reduces the number of brain T regulatory (Treg) cells) in acute or temporary conditions. In another embodiment, the agent reduces the number of brain Treg cells chronically, especially in the case of progressive, recurrent, or degenerative disease. In one embodiment, the agent may be administered simultaneously, or in another embodiment, it may be administered in a staggered fashion. In one embodiment, the staggered fashion may be dictated by the stage or phase of the disease.

In one embodiment, the composition of the invention further comprises an agent which suppresses neurotoxic inflammatory brain responses. In some embodiments, the phrase “neurotoxic inflammatory brain responses” refers to inflammation in the brain which results in tissue necrosis, or in some embodiments, elaboration of inflammatory mediators, or in some embodiments, extravasation of inflammatory cells, edema, or induction of heat shock proteins. In some embodiments, the phrase “neurotoxic inflammatory brain responses” refers to non-specific immune responses in the brain. In other embodiments, the phrase “neurotoxic inflammatory brain responses” refers to an innate response. In some embodiments, the phrase “neurotoxic inflammatory brain responses” refers to an acute inflammatory response, such as during infection, and in other embodiments, the phrase refers to a chronic response, such as in a neurodegenerative condition. In other embodiments, the phrase “neurotoxic inflammatory brain responses” refers to release of immune mediators. In one embodiment the agent suppressing a neurotoxic inflammatory brain response down-regulates or abrogates expression of any of the elements described herein, which contribute to a neurotoxic inflammatory brain response. In some embodiments, the agent which suppresses a neurotoxic inflammatory brain response directly, or in some embodiments, indirectly, interferes with expression or function of an element or mediator of a neurotoxic inflammatory brain response.

In some embodiments, neurotoxic inflammatory brain responses are marked by the stimulation or increased expression/production of tumor necrosis factor α, interleukin 1, interleukin 6, heat shock proteins, influx of inflammatory cells such as neutrophils, monocytes, and others. In some embodiments, neurotoxic inflammatory brain responses are marked by the stimulation or increased expression/production of ICAM-1, VCAM, and E-selectin facilitating influx of inflammatory cells. In some embodiments, neurotoxic inflammatory brain responses are marked by the stimulation or increased expression/production of caspases, for example caspase-3. Agents which suppress any or multiple aspects described are to be considered as part of this invention.

In another embodiment, the agent helps to stimulate and control neurogenesis. In another embodiment, the agent induces differentiation of progenitor cells to form neurons. One type of agent suitable for the present invention is nucleic acids which encode growth factor products, in particular neurotrophic growth factors. Such nucleic acids include, but are not limited to, the nucleic acid encoding BDNF, the neurotrophins NT-3 and NT-4/NT-5, insulin-like growth factor, nerve growth factor (NGF), the recently identified neurotrophic family of factors designated “NNT”. In one embodiment, the agent is a neurotrophin. In one embodiment, the neurotrophin is brain-derived neurotrophic factor (BDNF). In another embodiment, the neurotrophin is neurotrophin-3 (NT-3). In another embodiment, the neurotrophin is neurotrophin-4 (NT-4).

This invention encompasses administration of compounds as described herein or compositions comprising the same, for treating diseases and disorders related to degenerative or atrophic conditions of the CNS.

Drug delivery to the CNS may, in some embodiments of this invention, be by systemic administration, injection into CSF pathways, or direct injection into the brain, and in some embodiments, the compositions of this invention are formulated for any of these routes. In one embodiment, the compositions of the present invention are administered by systemic or direct administration into the CNS for targeted action in the CNS, and in some embodiments, the compositions of this invention are formulated for any of these routes. In one embodiment, the composition as set forth herein is formulated for brain-specific delivery, and in some embodiments, the compositions of this invention are formulated for any of these routes. In one embodiment, strategies for drug delivery to the brain include osmotic and chemical opening of the blood-brain barrier (BBB), as well as the use of transport or carrier systems, enzymes, and receptors that control the penetration of molecules in the blood-brain barrier endothelium, and in some embodiments, the compositions of this invention are formulated for any of these routes. In another embodiment, receptor-mediated transcytosis can transport peptides and proteins across the BBB, and in some embodiments, the compositions of this invention are formulated for any of these routes. In other embodiments, strategies for drug delivery to the brain involve bypassing the BBB, and in some embodiments, the compositions of this invention are formulated for any of these routes. In some embodiments, various pharmacological agents are used to open the BBB, and in some embodiments, the compositions of this invention are formulated for any of these routes.

In one embodiment, the route of administration may be directed to an organ or system that is affected by neurodegenerative conditions. For example, compounds may be administered topically. In another embodiment, the route of administration may be directed to a different organ or system than the one that is affected by neurodegenerative conditions. For example, compounds may be administered parenterally to treat neurodegenerative conditions. Thus, the present invention provides for the use of various dosage forms suitable for administration using any of the routes listed herein, and any routes which avail the CNS of such materials, as will be appreciated by one skilled in the art.

In some embodiments, the compositions/agents of the invention are specifically formulated such that they cross the blood-brain barrier (BBB). One example of such formulation comprises the use of specialized liposomes, which may be manufactured, for example as described U.S. Pat. Nos. 4,522,811; 5,374,548; and 5,399,331. In some embodiments, the liposomes comprise one or more moieties which are selectively transported into specific cells or organs (“targeting moieties” or “targeting groups” or “transporting vectors”), thus providing targeted drug delivery (see, e.g., V. V. Ranade J. Clin. Phamacol. 29, 685 (1989) fully incorporated by reference herein). In some embodiments the agents are linked to targeting groups that facilitate penetration of the blood brain barrier. In some embodiments, to facilitate transport of agents of the invention across the BBB, they may be coupled to a BBB transport vector (see, for example, Bickel et al., Adv. Drug Delivery Reviews 46, 247-79 (2001) fully incorporated by reference herein). In some embodiments, transport vectors include cationized albumin or the OX26 monoclonal antibody to the transferrin receptor; which undergo absorptive-mediated and receptor-mediated transcytosis through the BBB, respectively. Natural cell metabolites that may be used as targeting groups include, inter alia, putrescine, spermidine, spermine, or DHA. Other exemplary targeting moieties include folate or biotin (see, e.g., U.S. Pat. No. 5,416,016 fully incorporated by reference herein); mannosides (Umezawa et al., Biochem. Biophys. Res. Commun. 153, 1038 (1988) fully incorporated by reference herein); antibodies (P. G. Bloeman et al., FEBS Lett. 357, 140 (1995); M. Owais et al., Antimicrob. Agents Chemother. 39, 180 (1995)); surfactant protein A receptor (Briscoe et al., Am. J. Physiol. 1233, 134 (1995 fully incorporated by reference herein)); gp120 (Schreier et al., J. Biol. Chem. 269, 9090 (1994)); see also, K. Keinanen and M. L. Laukkanen, FEBS Lett. 346, 123 (1994); J. J. Killion and I. J. Fidler, Immunomethods 4, 273 (1994) all of which are fully incorporated by reference herein).

In some embodiments, BBB transport vectors that target receptor-mediated transport systems into the brain comprise factors such as insulin, insulin-like growth factors (“IGF-I,” and “IGF-II”), angiotensin II, atrial and brain natriuretic peptide (“ANP,” and “BNP”), interleukin I (“IL-1”) and transferrin. Monoclonal antibodies to the receptors that bind these factors may also be used as BBB transport vectors. BBB transport vectors targeting mechanisms for absorptive-mediated transcytosis include cationic moieties such as cationized LDL, albumin or horseradish peroxidase coupled with polylysine, cationized albumin or cationized immunoglobulins. Small basic oligopeptides such as the dynorphin analogue E-2078 and the ACTH analogue ebiratide may also cross the brain via absorptive-mediated transcytosis and are potential transport vectors. Other BBB transport vectors target systems for transporting nutrients into the brain. Examples of such BBB transport vectors include hexose moieties, e.g., glucose and monocarboxylic acids, e.g., lactic acid and neutral amino acids, e.g., phenylalanine and amines, e.g., choline and basic amino acids, e.g., arginine, nucleosides, e.g., adenosine and purine bases, e.g., adenine, and thyroid hormone, e.g., triiodothyridine. Antibodies to the extracellular domain of nutrient transporters may also be used as transport vectors. Other possible vectors include angiotensin II and ANP, which may be involved in regulating BBB permeability.

In some cases, the bond linking the therapeutic agent to the transport vector may be cleaved following transport into the brain in order to liberate the biologically active agent. Exemplary linkers include disulfide bonds, ester-based linkages, thioether linkages, amide bonds, acid-labile linkages, and Schiff base linkages. Avidin/biotin linkers, in which avidin is covalently coupled to the BBB drug transport vector, may also be used. Avidin itself may be a drug transport vector. Transcytosis, including receptor-mediated transport of compositions across the blood brain barrier, may also be suitable for the agents of the invention. Transferrin receptor-mediated delivery is disclosed in U.S. Pat. Nos. 5,672,683; 5,383,988; 5,527,527; 5,977,307; and 6,015,555, all of which are fully incorporated herein by reference. Transferrin-mediated transport is also known. P. M. Friden et al., Pharmacol. Exp. Ther. 278, 1491-98 (1996); H. J. Lee, J. Pharmacol. Exp. Ther. 292, 1048-52 (2000) all of which are fully incorporated herein by reference. EGF receptor-mediated delivery is disclosed in Y. Deguchi et al., Bioconjug. Chem. 10, 32-37 (1999), and transcytosis is described in A. Cerletti et al., J. Drug Target. 8, 435-46 (2000) all of which are fully incorporated herein by reference. Insulin fragments have also been used as carriers for delivery across the blood brain barrier. M. Fukuta et al., Pharm. Res. 11. 1681-88 (1994). Delivery of agents via a conjugate of neutral avidin and cationized human albumin has also been described. Y. S. Kang et al., Pharm. Res. 1, 1257-64 (1994) all of which are fully incorporated herein by reference. Other modifications in order to enhance penetration of the agents of the invention across the blood brain barrier may be accomplished using methods and derivatives known in the art. For example, U.S. Pat. No. 6,024,977 discloses covalent polar lipid conjugates for targeting to brain and central nervous system. U.S. Pat. No. 5,017,566 discloses cyclodextrin derivatives comprising inclusion complexes of lipoidal forms of dihydropyridine redox targeting moieties. U.S. Pat. No. 5,023,252 discloses the use of pharmaceutical compositions comprising a neurologically active drug and a compound for facilitating transport of the drug across the blood-brain barrier including a macrocyclic ester, diester, amide, diamide, amidine, diamidine, thioester, dithioester, thioamide, ketone or lactone. U.S. Pat. No. 5,024,998 discloses parenteral solutions of aqueous-insoluble drugs with cyclodextrin derivatives. U.S. Pat. No. 5,039,794 discloses the use of a metastatic tumor-derived egress factor for facilitating the transport of compounds across the blood-brain barrier. U.S. Pat. No. 5,112,863 discloses the use of N-acyl amino acid derivatives as antipsychotic drugs for delivery across the blood-brain barrier. U.S. Pat. No. 5,124,146 discloses a method for delivery of therapeutic agents across the blood-brain barrier at sites of increase permeability associated with brain lesions. U.S. Pat. No. 5,153,179 discloses acylated glycerol and derivatives for use in a medicament for improved penetration of cell membranes. U.S. Pat. No. 5,177,064 discloses the use of lipoidal phosphonate derivatives of nucleoside antiviral agents for delivery across the blood-brain barrier. U.S. Pat. No. 5,254,342 discloses receptor-mediated transcytosis of the blood-brain barrier using the transferrin receptor in combination with pharmaceutical compounds that enhance or accelerate this process. U.S. Pat. No. 5,258,402 discloses treatment of epilepsy with imidate derivatives of anticonvulsive sulfamate. U.S. Pat. No. 5,270,312 discloses substituted piperazines as central nervous system agents. U.S. Pat. No. 5,284,876 discloses fatty acid conjugates of dopamine drugs. U.S. Pat. No. 5,389,623 discloses the use of lipid dihydropyridine derivatives of anti-inflammatory steroids or steroid sex hormones for delivery across the blood-brain barrier. U.S. Pat. No. 5,405,834 discloses prodrug derivatives of thyrotropin releasing hormone. U.S. Pat. No. 5,413,996 discloses acyloxyalkyl phosphonate conjugates of neurologically-active drugs for anionic sequestration of such drugs in brain tissue. U.S. Pat. No. 5,434,137 discloses methods for the selective opening of abnormal brain tissue capillaries using bradykinin infused into the carotid artery. U.S. Pat. No. 5,442,043 discloses a peptide conjugate between a peptide having a biological activity and incapable of crossing the blood-brain barrier and a peptide which exhibits no biological activity and is capable of passing the blood-brain barrier by receptor-mediated endocytosis. U.S. Pat. No. 5,466,683 discloses water soluble analogues of an anticonvulsant for the treatment of epilepsy. U.S. Pat. No. 5,525,727 discloses compositions for differential uptake and retention in brain tissue comprising a conjugate of a narcotic analgesic and agonists and antagonists thereof with a lipid form of dihydropyridine that forms a redox salt upon uptake across the blood-brain barrier that prevents partitioning back to the systemic circulation all of which are fully incorporated herein by reference.

It is to be understood that reference to any publication, patent application or issued patent is to be considered as fully incorporated herein by reference in its entirety.

Nitric oxide is a vasodilator of the peripheral vasculature in normal tissue of the body. Increasing generation of nitric oxide by nitric oxide synthase causes vasodilation without loss of blood pressure. The blood-pressure-independent increase in blood flow through brain tissue increases cerebral bioavailability of blood-born compositions. This increase in nitric oxide may be stimulated by administering L-arginine. As nitric oxide is increased, cerebral blood flow is consequently increased, and drugs in the blood stream are carried along with the increased flow into brain tissue. Therefore, L-arginine may be used in the pharmaceutical compositions of the invention to enhance delivery of agents to brain tissue after introducing a pharmaceutical composition into the blood stream of the subject substantially contemporaneously with a blood flow enhancing amount of L-arginine, as described in WO 00/56328.

Still further examples of modifications that enhance penetration of the blood brain barrier are described in International (PCT) Application Publication Number WO 85/02342, which discloses a drug composition comprising a glycerolipid or derivative thereof. PCT Publication Number WO 089/11299 discloses a chemical conjugate of an antibody with an enzyme which is delivered specifically to a brain lesion site for activating a separately-administered neurologically-active prodrug. PCT Publication Number WO 91/04014 discloses methods for delivering therapeutic and diagnostic agents across the blood-brain barrier by encapsulating the drugs in liposomes targeted to brain tissue using transport-specific receptor ligands or antibodies. PCT Publication Number WO 91/04745 discloses transport across the blood-brain barrier using cell adhesion molecules and fragments thereof to increase the permeability of tight junctions in vascular endothelium. PCT Publication Number WO 91/14438 discloses the use of a modified, chimeric monoclonal antibody for facilitating transport of substances across the blood-brain barrier. PCT Publication Number WO 94/01131 discloses lipidized proteins, including antibodies. PCT Publication Number WO 94/03424 discloses the use of amino acid derivatives as drug conjugates for facilitating transport across the blood-brain barrier. PCT Publication Number WO 94/06450 discloses conjugates of neurologically-active drugs with a dihydropyridine-type redox targeting moiety and comprising an amino acid linkage and an aliphatic residue. PCT Publication Number WO 94/02178 discloses antibody-targeted liposomes for delivery across the blood-brain barrier. PCT Publication Number WO 95/07092 discloses the use of drug-growth factor conjugates for delivering drugs across the blood-brain barrier. PCT Publication Number WO 96/00537 discloses polymeric microspheres as injectable drug-delivery vehicles for delivering bioactive agents to sites within the central nervous system. PCT Publication Number WO 96/04001 discloses omega-3-fatty acid conjugates of neurologically-active drugs for brain tissue delivery. PCT WO 96/22303 discloses fatty acid and glycerolipid conjugates of neurologically-active drugs for brain tissue delivery. In one embodiment, the active compound can be delivered in a vesicle, for example, a liposome. In another embodiment, the active compound can be delivered as a nanoparticle. In one embodiment, delivery may be specifically targeted to the CNS. In another embodiment, the active compounds may be delivered by any method described herein. The compositions of this invention may comprise ingredients known to the skilled artisan to be useful in formulating compositions for administration to a subject. In some embodiments, the compositions will comprise pharmaceutically acceptable carriers or diluents. n some embodiments, the phrase “pharmaceutically acceptable carriers or diluents” may comprise a solid carrier or diluent for solid formulations, a liquid carrier or diluent for liquid formulations, or mixtures thereof.

In some embodiments, the compositions/agents of the invention comprise a “piggyback mechanism” to deliver specific desirable agents, or combinations thereof to the CNS, i.e. to ensure that they cross the blood-brain barrier (BBB).

In some embodiments, as exemplified herein, Aβ immunization resulted in Aβ-specific T cells, which in turn specifically extravasate to sites of Aβ deposition in the CNS. Such cells in turn, may function as delivery vehicles for the soluble factors that they secrete, to sites of Aβ deposition. In some embodiments, the soluble factors may comprise cytokines, growth factors, or other desirable molecules. In some embodiments, such Aβ-specific T cell delivery vehicles may be adoptively transferred to a desirable host, wherein the cells are autologous, i.e. educated ex-vivo and reintroduced, or in some embodiments, such cells are allogeneic, etc.

In some embodiments, this invention provides compositions comprising an agent which specifically increases brain interferon-γ levels, wherein the agent may comprise an Aβ-specific T cell, as herein described. In some embodiments, such compositions may further comprise another agent which specifically increases brain interferon-γ levels, as herein described, an agent, which reduces or abrogates brain specific T reg cells, and/or an agent which reduces neurotoxic inflammatory brain responses, as herein described. In some embodiments, this invention provides for the sue of such compositions or kits comprising such agents, for the treatment of neurodegenerative diseases as herein described.

It is to be understood that this invention provides compositions, kits and uses of any combination of any agents as described herein, and such combinations represent embodiments of this invention.

Solid carriers/diluents include, but are not limited to, a gum, a starch (e.g., corn starch, pregeletanized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof.

Further, in another embodiment, the pharmaceutical compositions are administered as a suppository, for example a rectal suppository or a urethral suppository. Further, in another embodiment, the pharmaceutical compositions are administered by subcutaneous implantation of a pellet. In a further embodiment, the pellet provides for controlled release of an agent over a period of time. In yet another embodiment, the pharmaceutical compositions are administered in the form of a capsule.

The compositions as set forth herein may be in a form suitable for intracranial administration. In some embodiments, direct methods to introduce therapeutic agents into the brain substance include the use of devices and needles, such as in the case of intrathecal and intracerebroventricular delivery. In other embodiments, direct methods to introduce therapeutic agents into the brain substance include the use of magnets coupled to the composition of the invention for site-directed delivery. In other embodiments, direct methods to introduce therapeutic agents into the brain substance include the use of heat-activated compounds coupled to the composition of the invention for site-directed delivery. In one embodiment, delivery of the agent to the CNS is a function of its ability to access a relevant target site within the CNS.

The compositions as set forth herein may be in a form suitable for intransal administration. In some embodiments, intranasal delivery insures CNS delivery, upon crossing the olfactory nerves, the trigeminal nerves, or both. Intranasal delivery does not require any modification of the therapeutic agents and does not require that drugs be coupled with any carrier like in the case of drug delivery across the BBB. The olfactory neural pathway provides two pathways across the BBB. The intraneuronal pathway involves axonal transport and requires hours to days for drugs to reach different brain regions, while an extraneuronal pathway into the brain relies on bulk flow transport through perineural channels, which deliver drugs directly to the brain parenchymal tissue and/or CSF, and allows therapeutic agents to reach the CNS within minutes. In some embodiments, intranasal delivery is via the intraneuronal pathway. In other embodiments, intranasal delivery is via the extraneuronal pathway. In another embodiment, intransal delivery is via a combination of the intraneuronal and extraneuronal pathways.

For intranasal administration or application by inhalation, solutions or suspensions of the compounds mixed and aerosolized or nebulized in the presence of the appropriate carrier suitable. Such an aerosol may comprise any agent described herein.

In one embodiment, the route of administration may be parenteral, or a combination thereof. In another embodiment, the route may be intra-ocular, conjunctival, topical, transdermal, intradermal, subcutaneous, intraperitoneal, intravenous, intra-arterial, vaginal, rectal, intratumoral, parcanceral, transmucosal, intramuscular, intravascular, intraventricular, intracranial, inhalation (aerosol), nasal aspiration (spray), intranasal (drops), sublingual, oral, aerosol or suppository or a combination thereof. In one embodiment, the dosage regimen will be determined by skilled clinicians, based on factors such as exact nature of the condition being treated, the severity of the condition, the age and general physical condition of the patient, body weight, and response of the individual patient, etc.

For parenteral application, particularly suitable are injectable, sterile solutions, preferably oily or aqueous solutions, as well as suspensions, emulsions, or implants, including suppositories and enemas. Ampoules are convenient unit dosages. Such a suppository may comprise any agent described herein.

Sustained or directed release compositions can be formulated, e.g., liposomes or those wherein the active compound is protected with differentially degradable coatings, e.g., by microencapsulation, multiple coatings, etc. Such compositions may be formulated for immediate or slow release. It is also possible to freeze-dry the new compounds and use the lyophilisates obtained, for example, for the preparation of products for injection.

For liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil.

Solid carriers/diluents include, but are not limited to, a gum, a starch (e.g., corn starch, pregeletanized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof.

In one embodiment, compositions of this invention are pharmaceutically acceptable. In one embodiment, the term “pharmaceutically acceptable” refers to any formulation which is safe, and provides the appropriate delivery for the desired route of administration of an effective amount of at least one compound for use in the present invention. This term refers to the use of buffered formulations as well, wherein the pH is maintained at a particular desired value, ranging from pH 4.0 to pH 9.0, in accordance with the stability of the compounds and route of administration.

In one embodiment, a composition of or used in the methods of this invention may be administered alone or within a composition. In another embodiment, compositions of this invention admixture with conventional excipients, i.e. pharmaceutically acceptable organic or inorganic carrier substances suitable for parenteral, enteral (e.g., oral) or topical application which do not deleteriously react with the active compounds may be used. In one embodiment, suitable pharmaceutically acceptable carriers include but are not limited to water, salt solutions, alcohols, gum arabic, vegetable oils, benzyl alcohols, polyethylene glycols, gelatine, carbohydrates such as lactose, amylose or starch, magnesium stearate, talc, silicic acid, viscous paraffin, white paraffin, glycerol, alginates, hyaluronic acid, collagen, perfume oil, fatty acid monoglycerides and diglycerides, pentaerythritol fatty acid esters, hydroxy methylcellulose, polyvinyl pyrrolidone, etc. In another embodiment, the pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. In another embodiment, they can also be combined where desired with other active agents, e.g., vitamins.

In one embodiment, the therapeutic compositions of the present invention may comprise the composition of this invention and one or more additional compounds effective in preventing or treating neurodegenerative conditions. In some embodiments, the additional compound may comprise an immunomodulating compound.

In one embodiment, the immunomodulating agent is an anti-inflammatory agent. In one embodiment, the anti-inflammatory agent is a non-steroidal anti-inflammatory agent. In one embodiment, the non-steroidal anti-inflammatory agent is a cox-1 inhibitor. In one embodiment, the non-steroidal anti-inflammatory agent is a cox-2 inhibitor. In one embodiment, the non-steroidal anti-inflammatory agent is a cox-1 and cox-2 inhibitor. In some embodiments, non-steroidal anti-inflammatory agents include but are not limited to aspirin, salsalate, diflunisal, ibuprofen, fenoprofen, flubiprofen, fenamate, ketoprofen, nabumetone, piroxicam, naproxen, diclofenac, indomethacin, sulindac, tolmetin, etodolac, ketorolac, oxaprozin, or celecoxib. In one embodiment, the anti-inflammatory agent is a steroidal anti-inflammatory agent. In one embodiment, the steroidal anti-inflammatory agent is a corticosteroid.

In general, the doses utilized for the above described purposes will vary, but will be in an effective amount to exert the desired effect, as determined by a clinician of skill in the art. As used herein, the term “pharmaceutically effective amount” refers to an amount of a compound as described herein, which will produce the desired alleviation in symptoms or other desired phenotype in a patient.

In one embodiment of the invention, the concentrations of the compounds will depend on various factors, including the nature of the condition to be treated, the condition of the patient, the route of administration and the individual tolerability of the compositions.

In some embodiments, any of the compositions of this invention will comprise a compound, in any form or embodiment as described herein. In some embodiments, any of the compositions of this invention will consist of a compound, in any form or embodiment as described herein. In some embodiments, any of the compositions of this invention will consist essentially of a compound, in any form or embodiment as described herein. In some embodiments, the term “comprise” refers to the inclusion of the indicated active agent, such as the compound of this invention, as well as inclusion of other active agents, and pharmaceutically acceptable carriers, excipients, emollients, stabilizers, etc., as are known in the pharmaceutical industry. In some embodiments, the term “consisting essentially of” refers to a composition whose only active ingredient is the indicated active ingredient, however, other compounds may be included which are for stabilizing, preserving, etc. the formulation, but are not involved directly in the therapeutic effect of the indicated active ingredient. In some embodiments, the term “consisting essentially of” may refer to components which facilitate the release of the active ingredient. In some embodiments, the term “consisting” refers to a composition, which contains the active ingredient and a pharmaceutically acceptable carrier or excipient.

It will be appreciated that the actual preferred amounts of active compound in a specific case will vary according to the specific compound being utilized, the particular compositions formulated, the mode of application, and the particular conditions and organism being treated. Dosages for a given host can be determined using conventional considerations, e.g., by customary comparison of the differential activities of the subject compounds and of a known agent, e.g., by means of an appropriate, conventional pharmacological protocol.

In one embodiment, the compounds of the invention may be administered acutely for acute treatment of temporary conditions, or may be administered chronically, especially in the case of progressive, recurrent, or degenerative disease. In one embodiment, one or more compounds of the invention may be administered simultaneously, or in another embodiment, they may be administered in a staggered fashion. In one embodiment, the staggered fashion may be dictated by the stage or phase of the disease.

Parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of petroleum, animal, vegetable, or synthetic origin, for example, peanut oil, soybean oil, mineral oil, olive oil, sunflower oil, and fish-liver oil.

In addition, the compositions may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmelose sodium, crospovidone, guar gum, sodium starch glycolate), buffers (e.g., Tris-HCl, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g., hydroxypropyl cellulose, hyroxypropylmethyl cellulose), viscosity increasing agents (e.g., carbomer, colloidal silicon dioxide, ethyl cellulose, guar gum), sweeteners (e.g., aspartame, citric acid), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates) and/or adjuvants.

In one embodiment, the pharmaceutical compositions provided herein are controlled-release compositions, i.e. compositions in which the anti-estrogen compound is released over a period of time after administration. Controlled- or sustained-release compositions include formulation in lipophilic depots (e.g., fatty acids, waxes, oils). In another embodiment, the composition is an immediate-release composition, i.e. a composition in which all of the compound is released immediately after administration. In one embodiment, the controlled- or sustained-release compositions of the invention are administered as a single dose. In another embodiment, compositions of the invention are administered as multiple doses, over a varying time period of minutes, hours, days, weeks, months or more. In another embodiment, compositions of the invention are administered during periods of acute disease. In another embodiment, compositions of the invention are administered during periods of chronic disease. In another embodiment, compositions of the invention are administered during periods of remission. In another embodiment, compositions of the invention are administered prior to development of gross symptoms.

In yet another embodiment, the pharmaceutical composition can be delivered in a controlled release system. For example, the agent may be administered using intravenous infusion, an implantable osmotic pump, a transdermal patch, liposomes, or other modes of administration. In one embodiment, a pump may be used. In another embodiment, polymeric materials can be used. In yet another embodiment, a controlled release system can be placed in proximity to the therapeutic target, i.e., the brain, thus requiring only a fraction of the systemic dose. In another embodiment, the controlled-release system may be any controlled release system known in the art.

The compositions may also include incorporation of the active material into or onto particulate preparations of polymeric compounds such as polylactic acid, polglycolic acid, hydrogels, etc., or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance.

Also comprehended by the invention are particulate compositions coated with polymers (e.g., poloxamers or poloxamines) and the compound coupled to antibodies directed against tissue-specific receptors, ligands or antigens or coupled to ligands of tissue-specific receptors.

Also comprehended by the invention are compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection than do the corresponding unmodified compounds. Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The preparation of pharmaceutical compositions that contain an active component, for example by mixing, granulating, or tablet-forming processes, is well understood in the art. The active therapeutic ingredient is often mixed with excipients that are pharmaceutically acceptable and compatible with the active ingredient. For oral administration, the compound is mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. For parenteral administration, the compound is converted into a solution, suspension, or emulsion, if desired with the substances customary and suitable for this purpose, for example, solubilizers or other substances.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For use in medicine, the salts are pharmaceutically acceptable salts. Other salts may, however, be useful in the preparation of the compounds according to the invention or of their pharmaceutically acceptable salts. Suitable pharmaceutically acceptable salts of the compounds of this invention include acid addition salts, which may, for example, be formed by mixing a solution of the compound according to the invention with a solution of a pharmaceutically acceptable acid such as hydrochloric acid, sulphuric acid, methanesulphonic acid, fumaric acid, maleic acid, succinic acid, acetic acid, benzoic: acid, oxalic acid, citric acid, tartaric acid, carbonic acid or phosphoric acid.

As defined herein, the term “contacting” means that the compound of the present invention is introduced into a subject receiving treatment, and the compound is allowed to come in contact with the NPC in vivo.

As used herein, the term “treating” includes preventive as well as disorder remitative treatment. As used herein, the terms “reducing”, “suppressing” and “inhibiting” have their commonly understood meaning of lessening or decreasing. As used herein, the term “progression” means increasing in scope or severity, advancing, growing or becoming worse. As used herein, the term “recurrence” means the return of a disease after a remission.

As used herein, the term “administering” refers to bringing a subject in contact with a compound of the present invention. As used herein, administration can be accomplished in vitro, i.e. in a test tube, or in vivo, i.e. in cells or tissues of living organisms, for example humans. In one embodiment, the present invention encompasses administering the compounds of the present invention to a subject.

Although the pharmaceutical compositions provided herein are principally directed to pharmaceutical compositions which are suitable for administration to humans, it will be understood by the skilled artisan that such compositions are generally suitable for administration to animals of all sorts. Modification of pharmaceutical composition suitable for administration to humans in order to render the compositions suitable for administration to various animals is well understood, and the ordinarily skilled veterinary pharmacologist can design and perform such modification with little, if any, experimentation. Subjects to which administration of the pharmaceutical compositions of the invention is contemplated include, but are not limited to, humans and other primates, and other mammals.

The composition of this invention may, in one embodiment, be provided as a kit useful for enhancing neurogeneis. In one embodiment, the kit of the invention comprises two different components for inducing and/or enhancing neurogenesis in a subject. In another embodiment, the kit further comprises agents which suppress innate, non-acquired immune responses in the brain. In one embodiment, the components of the kit are administered to the subject concurrently. In another embodiment, administration of the components to the subject is not concurrent. In another embodiment, administration of the components of the kit may be with a time lag of minutes, hours, days, months or any other period of time.

The agents, compositions and kits of this invention and methods of this invention are directed to treating a neurodegenerative disease.

In one embodiment, this invention provides a method for inducing or enhancing neurogenesis in a subject, said method comprising administering to a subject a composition comprising:
a) an agent which increases brain levels of interferon-γ; and
b) an agent which reduces the number of T regulatory (Treg) cells.

In some embodiments, this invention provides a method for inducing or enhancing neurogenesis in a subject, comprising administering an agent which reduces the number of T regulatory (Treg) cells to the subject. In some embodiments, the method comprises administering an agent which increases brain levels of interferon-γ. In some embodiments, the agent which increases brain levels of interferon-γ also concurrently reduces the number of Tregs. In some embodiments, the agent, which reduces the number of Tregs increases brain levels of interferon-γ. In some embodiments, a single agent accomplishing both tasks is utilized.

In another embodiment, the method further comprises administering an agent, which suppresses neurotoxic inflammatory brain responses.

In one embodiment, the neurodegenerative disease or disorder comprises an injury, disease, disorder or condition of the central nervous system (CNS). In another embodiment, the neurodegenerative disease or disorder comprises Alzheimer's disease, Parkinson's disease, Huntington's disease, multiple sclerosis, diabetic neuropathy or amyotrophic lateral sclerosis (ALS). In another embodiment, the neurodegenerative disease or disorder comprises spinal cord injury, closed head injury, blunt trauma, penetrating trauma, hemorrhagic stroke, ischemic stroke, cerebral ischemia, optic nerve injury, or injury caused by tumor excision. In another embodiment, the subject is at risk for a neurodegenerative disease or disorder.

In one embodiment, the neurodegenerative disease or disorder comprises epilepsy, amnesia, anxiety, hyperalgesia, psychosis, seizures, oxidative stress, opiate tolerance and dependence, a psychosis or psychiatric disorder comprising an anxiety disorder, a mood disorder, schizophrenia or a schizophrenia-related disorder, drug use or dependence or withdrawal, or a memory loss or cognitive disorder.

In another embodiment, neurodegenerative disease or disorder comprises facial nerve (Bell's) palsy, glaucoma, Alper's disease, Batten disease, Cockayne syndrome, Guillain-Barré syndrome, Lewy body disease, Creutzfeld-Jakob disease, or a peripheral neuropathy such as a mononeuropathy or polyneuropathy comprising adrenomeloneuropathy, alcoholic neuropathy, amyloid neuropathy or polyneuropathy, axonal neuropathy, chronic sensory ataxic neuropathy associated with Sjogren's syndrome, diabetic neuropathy, an entrapment neuropathy, nerve compression syndrome, carpal tunnel syndrome, a nerve root compression that may follow cervical or lumbar intervertebral disc herniation, giant axonal neuropathy, hepatic neuropathy, ischemic neuropathy, nutritional polyneuropathy due to vitamin deficiency, malabsorption syndromes or alcoholism, porphyric polyneuropathy, a toxic neuropathy caused by organophosphates, uremic polyneuropathy, a neuropathy associated with a disease or disorder comprising acromegaly, ataxia telangiectasia, Charcot-Marie-Tooth disease, chronic obstructive pulmonary diseases, Fabry's disease, Friedreich ataxia, Guillain-Barre syndrome, hypoglycemia, IgG or IgA monoclonal gammopathy (non-malignant or associated with multiple myeloma or with osteosclerotic myeloma), lipoproteinemia, polycythemia vera, Refsum's syndrome, Reye's syndrome or Sjogren-Larrson syndrome, a polyneuropathy associated with various drugs, with hypoglycemia, with infections as HIV infection, or with cancer.

Infection of the central nervous system, such as, for example, in meningitis, or encephalomyelitis, is often accompanied by an inflammatory response, which is destructive to the tissue. Such scenarios benefit, in one embodiment, by the methods/compositions/kits of this invention.

In one embodiment, this invention provides for methods of treatment of diseases or disorders involving the central nervous system, including, inter alia, pain, myasthenia gravis (MG), fronto-temporal dementia (FTD), stroke, traumatic brain injury, HIV-associated dementia, encephalomyelitis, chronic inflammatory demyelinating polyneuropathy, cerebral ischemia-induced injury, age-related retinal degeneration, or any combination thereof.

In another embodiment, this invention provides for methods of treatment of diseases and disorders related to degenerative or atrophic conditions, which may include, but are not limited to, autoimmune diseases and cerebrovascular and neurodegenerative diseases or disorders in the central and peripheral nervous system.

In another embodiment, the invention provides methods for treatment of central nervous system damage as a result of an inflammatory response. The phrase “central nervous system damage” or “CNS damage” refers, in some embodiments, to the result of a disease process or injury that is characterized by destruction of, or harm to, cells of the brain or the spinal cord, such that the normal motor and sensory control function of the brain or spinal cord is disrupted. CNS damage shall be understood to encompass, for example, the result of an acute traumatic break or injury of the spine that completely or partially severs the spinal cord, the result of a stroke, the result of chronic disease such as multiple sclerosis, Huntington's Disease, Alzheimer's disease, amyotrophic lateral sclerosis (ALS) and neurodegeneration of aging, and the result of cancerous tumors forming within the central nervous system. A subject suffering from CNS damage is deemed to also be suffering from at least a partial disruption of motor or of sensory function, or of both motor and sensory function as a result of the CNS damage.

As used interchangeably herein, the terms “spinal cord injury” or “SCI” refer to a specific instance of CNS damage characterized by complete or partial destruction of the spinal cord at one or more sites, which may result from acute trauma to the spine or from a disease process.

As described hereinbelow, the methods of this invention may be used to treat such damage to the nervous system, and may be effective in some embodiments, in evoking patterned movement in a subject suffering from CNS damage, such as a motor complete subject, at least partially restoring previously lost CNS function and regenerating neural cells in or around a site of CNS damage, in other embodiments. In other embodiments, the methods of this invention, and compositions/compounds used thereof, at least partially restore motor and sensory function in individuals suffering from CNS damage, and may, in other embodiments, be useful in those individuals with such extensive CNS damage that recovery of any function requiring the activity of the lost or damaged neurons was previously thought impossible.

In one embodiment, “preventing, or treating” refers to any one or more of the following: delaying the onset of symptoms, reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, preventing relapse to a disease, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics. In one embodiment, “treating” refers to both therapeutic treatment and prophylactic or preventive measures, wherein the object is to prevent or lessen the targeted pathologic condition or disorder as described hereinabove.

In another embodiment, “symptoms” may be any manifestation of a disease or pathological condition as described hereinabove.

In one embodiment, methods of the present invention involve treating a subject by, inter alia, controlling the expression, production, and activity of cytokines, chemokines and interleukins; anti-oxidant therapy; anti-endotoxin therapy or any combination thereof.

The administration mode of the compounds and compositions of the present invention, timing of administration and dosage, i.e. the treatment regimen, will depend on the type and severity of the injury, disease or disorder, and the age and condition of the subject. In one embodiment, the compounds and compositions may be administered concomitantly. In another embodiment, the compounds and compositions may be administered at time intervals of seconds, minutes, hours, days, weeks or more.

In one embodiment, the method comprises administering the agents in a composition in a form suitable for administration via an intracranial route. In another embodiment, the composition is in a form suitable for administration via an intranasal route. In another embodiment, the method comprises administering the composition via an oral, intravenous, intraarterial, intramuscular, subcutaneous, parenteral, transmucosal, transdermal, or topical route.

In one embodiment, the method further comprises administering neurotrophins. In another embodiment, the neurotrophins comprise BDNF, NT-3 or NT4.

In one embodiment of the invention, the term “controlling” refers to inhibiting the production and action of the above mentioned factors in order to maintain their activity at the normal basal level and suppress their activation in pathological conditions.

In one embodiment, methods for corroborating induced and/or enhanced neurogenesis in a subject include, inter alia, CT scan, magnetic resonance imaging (MRI), and any other methods currently known or any methods to be developed in the future for visualizing soft body tissue in a living subject. In another embodiment, methods for corroborating induced and/or enhanced neurogenesis in a subject include immunological/histological methods such as, inter alia, enzyme-linked immunosorbant assay (ELISA). It is to be understood that induction of levels of interferon-γ may be measured locally and peripherally.

Tests for cognitive ability may be general intelligence tests and/or aptitude tests, wherein aptitude may be mechanical aptitude, clerical aptitude, or spatial aptitude. Tests may be tests of verbal comprehension, numerical ability, visual pursuit, visual speed and accuracy, space visualization, numerical reasoning, verbal reasoning, word fluency, manual speed and accuracy, symbolic reasoning, short-tem memory tests, or information processing tests. Tests for cognitive ability in rodents may be an eight-arm radial maze, a Barnes circular platform maze or a Morris water maze.

### EXAMPLES

### Materials And Experimental Methods

Mice. Mouse strains included C57BL6 and SJL mice. Mice of the APP-Tg J20 line on a C57BL6 background expressed APP under the platelet-derived growth factor (PDGF) promoter. Transgenic SJL mice expressed IFN-γ under the myelin basic protein (MBP) promoter. Homozygous IFN-γ-Tg mice were crossed with C57BL6 and APP-Tg mice to generate IFN-γ Tg and APP/IFN-γ double Tg B6SJLF1 mice, respectively. SJL mice were crossed with C57BL6 and APP Tg mice to generate wild-type and APP Tg B6SJLF1 mice, respectively. All the experiments described were performed using the B6SJLF1 genetic background.

Immunohistochemistry (1HC) BrdU administration. Long- and short-term injections of BrdU were used to estimate proliferation and differentiation of neuronal progenitor cells (NPCs) in the dentate gyrus (DG). In the long-term procedure, animals received daily intraperitoneal (i.p.) injections of BrdU (50 mg/kg of body weight per day) for eight consecutive days and were killed one day later. For the short-term procedure, mice were injected with BrdU for three days and killed on day 3, 2 h after the last injection.

Staining. Animals were given deep anesthesia with an overdose of pentobarbital and then cardially perfused with 10 ml of PBS, followed by 10 ml of 4% paraformaldehyde. Brains were then excised and incubated in 30% sucrose solution for 48 h at 4° C. The tissues were frozen in isopentane cooled by liquid nitrogen and stored at −80° C. IHC staining was conducted on free floating 40-μm sections that had been cut with a cryostat. For double-labeling of doublecortin (DCX) and BrdU, slices were pretreated with 2 M HCl at 37° C. for 30 min and neutralized with 0.1 M borate buffer (pH 8.5) for 10 min. Slices were incubated first for 1 h with primary antibody diluting buffer containing 0.3% Triton-X and then overnight at 4° C. in the presence of DCX and α-BrdU antibodies diluted 1:50 and 1:200, respectively. To label newly generated oligodendrocytes, tissues were double-labeled with anti-NG-2 diluted 1:50 and anti-BrdU for two days at 4° C. To determine the integrity of presynaptic terminals, cryostat sections (40 μm) were labeled with anti-synaptophysin using a M.O.M. kit™ according to the manufacturer's procedure. In short, tissues were blocked with blocking solution for 1 h at room temperature, rinsed briefly with PBS/Tween (0.05%), and incubated for 1 h with synaptophysin antibody. Alexa 488 and 546 diluted 1:500 were used as secondary antibodies. Where indicated, slices were counterstained with TO-PRO-3 iodide (642/661). All images were obtained using an Olympus FluoView FV1000 confocal microscope.

Image analysis Neurogenesis. Neurogenesis in the DG was evaluated by counting BrdU and DCX single- and double-positive cells in the DG. In each section, the whole DG (20 μm thick) was Z-scanned using the ×40 lens, and the composite of the Z-stack images was analyzed. Labeled cells in five sagittal sections evenly distributed throughout the hippocampus (lateral 0.6-1.8 mm, atlas of a C57BL/6J brain by K. Franklin and G. Paxinos) of each mouse were counted and averaged.

Synaptophysin. Images were taken using the ×100 objective and a ×2.7 digital zoom. Microscope setting was determined according to the non-transgenic wild-type tissues, and all the images in the experiment were taken under this microscope setting. Three regions were imaged and analyzed—CA1 and CA3 of the hippocampus and the molecular layer of the DG. For each region, three randomized images were obtained from three sections per mouse and analyzed using Volocity software. The synatophysin-stained area was quantified and expressed as percentage of the total image area.

Oligodendrogenesis. The number of newly generated oligodendrocytes was evaluated by counting BrdU/NG-2 double-positive cells in the hippocampus in three sagittal sections per mouse throughout the hippocampus (lateral 0.6-1.8 mm, atlas of a C57BL/6J brain by K. Franklin and G. Paxinos). In each section, BrdU/NG-2 cells were counted only in a 3D projection of the Z-stack images.

Quantitative PCR. Wild type and INF-γ Tg mice were anesthetized by i.p. injection of ketamine-xylazine and perfused with ice-cold PBS buffer. For each mouse, the brain was removed from the skull and the hippocampus-cortex was excised on ice. Total RNA was extracted with the Aurum Total RNA Mini Kit according to the manufacturer's instructions. Total RNA, 5 μg, was reverse transcribed using Bio-RT kit according to the manufacturer's instructions. The primers for real time (RT) PCR were designed using the Primer Express software and synthesized by Sigma.

Real time quantification of genes was performed using a SYBER-Green real-time RT-PCR assay (Invitrogen). For 20 μl of the RT-PCR reaction mixture, 125 ng of total cDNA were used. The samples were run in triplicate, and the level of expression of each gene was compared with the expression of beta-actin. Amplification, detection of specific gene products, and quantitative analysis were performed using an ABI PRISM 7500 sequence detection system.

MWM behavioral test for spatial learning and memory. For the acquisition phase, four trials per day were performed on five consecutive days. In each trial, the mouse was required to find a hidden platform located 1.5 cm below the water surface in a 1.1-m diameter pool. Various extramaze cues, i.e., black geometric images (squares, circles, and triangles) on the walls, the light, and the shelves around the pool served as visual reference points for the animals. A trial was initiated by placing each mouse in the water facing the pool wall at one of four starting points. The escape latency, i.e., the time required by the mouse to find the platform and climb onto it, was recorded for up to 60 s. Each mouse was allowed to remain on the platform for 20 s and was then moved from the maze to its home cage. If the mouse did not find the platform within 60 s, it was manually placed on the platform and returned to its home cage after 20 s. The interval between trials was 600 s. On day 6, the platform was removed from the pool, and each mouse was tested in a probe trial for 60 s to measure spatial bias. To create a standardizable recent-memory task, the MWM protocol was adapted so that the platform finding task was altered slightly, requiring the test mouse to distinguish between earlier memories and the most recent memory. After the mouse had learned to escape quickly and reliably onto the hidden platform at one location, the platform moved to a new location. Three locations were used one at a time. In such a procedure, the memory of earlier locations of the platform would therefore have to be selective for the most recently encoded location, an episodic-like component of the task. For the reversal phase, mice were subjected to four trials per day, for two consecutive days in each platform location. Data were recorded using an EthoVision automated tracking system.

Statistical Analysis. For the MWM paradigm, variables were analyzed by 3-way repeated measures analysis of variance (ANOVA) to compare effects within groups, in the four different trials, and the in the five different days (for the acquisition phase), or for the two different days for the reversal phase. For the probe trial phase, the statistical analyses were performed using one-way ANOVA. The areas under the curve (AUCs) were calculated by the trapezoidal rule.

### Example 1

### Kinetics of Brain IFN-γ mRNA Expression in IFN-γ Tg Mice

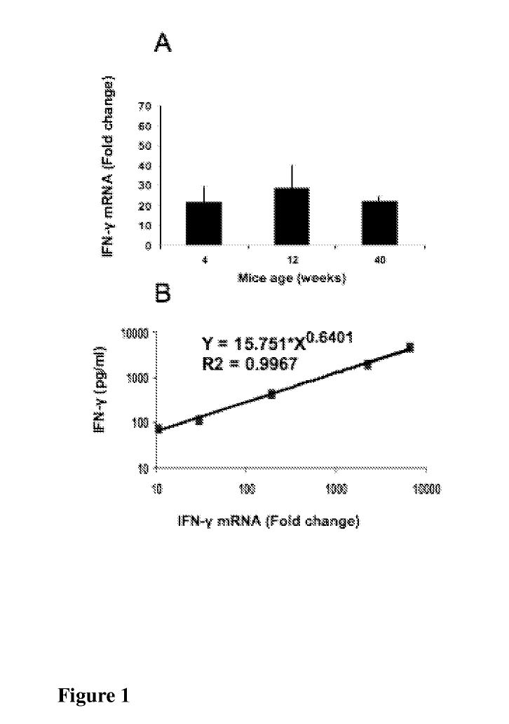

Expression of IFN-γ mRNA in the brains of 30-, 90-, and 270-day-old IFN-γ-expressing transgenic (Tg) mice and wild-type control mice was analyzed by quantitative PCR (qPCR). The amounts of IFN-γ at 1 month of age were 22±7.5 (SEM)-fold higher in IFN-γ Tg mice than those in wild-type control mice, with no significant differences at three and nine months of age (FIG. 1A). These clearly detectable amounts of IFN-γ mRNA in the brain did not cause tissue abnormalities, gliosis, or spontaneous immune infiltration in the CNS, but were sufficiently high to allow T-cell entry to sites of neuritic plaques upon Aβ immunization. Since these low concentrations of the IFN-γ protein could not be detected using brain-lysate ELISA or western blot analysis (data not shown), the amount of IFN-γ protein secreted from activated lymphocytes (grown in a serum-free medium) expressing equivalent amounts of IFN-γ mRNA to those expressed in brains of IFN-γ Tg mice was measured. As calculated from the correlation analysis shown in FIG. 1B, the RQs [28.8±11.66 (SEM, standard error mean) of IFN-γ mRNA obtained in brains of 3-month-old IFN-γ Tg mice corresponded to 125±34.57 (SEM) pg/ml IFN-γ. These amounts of IFN-γ were obtained with very weak lymphocyte stimulation and considered low compared to at least 10 ng/ml used in most in vitro assays.

### Example 2

### Neurogenesis is Increased in the DG of IFN-γ Tg Mice

To determine the effect of IFN-γ on neurogenesis, mice were injected with bromodeoxyuridine (BrdU), brain sections were immunolabeled with antibodies to BrdU and doublecortin (DCX), and the amounts of neuronal progenitor cells were quantified. A composite of Z-stack images taken in the DG demonstrating BrdU (green) and DCX (red) single color images and their colocalization in a 3D representation image is shown in FIG. 2A. At both three and nine months of age, the amounts of BrdU/DCX-positive cells were higher in the DGs of the IFN-γ Tg mice than in the wild-type mice (FIG. 2B). Stereological quantification of BrdU/DCX positive cells in the DG of three-month-old wild-type mice revealed an average of 41.47±3.48 (SEM) cells per section (FIG. 3C), or 4,548±693.4 (SEM) cells in the entire DG. As early as three months of age there were significantly higher amounts of both BrdU and BrdU/DCX populations in IFN-γ Tg mice than in wild-type controls (FIG. 2C). At nine months of age, there was a decline in BrdU- and DCX-immunolabeled cells in wild-type mice, as previously observed⁴, but this decrease was less marked in IFN-γ Tg mice (FIG. 2D). The amounts of BrdU/DCX cells in the hippocampus were higher, by 1.19- and 1.74-fold in the DGs of three- and nine-month-old IFN-γ Tg mice than in wild-type controls, respectively (FIG. 2C-D).

From the above data, it appeared that IFN-γ enhanced neurogenesis in young mice, but to a lesser extent than in older mice. Since neurogenesis is more intense in young mice, the eight-day BrdU injection protocol was replaced with a shorter protocol, in which mice were injected with BrdU for three days and killed 2 h after the last injection. At one month, numbers of BrdU- and BrdU/DCX-positive cells in the DG did not differ significantly between IFN-γ Tg and wild-type mice (FIG. 2E, left panel). At three months of age, IFN-γ enhanced neurogenesis though to a lower extent than that found for mice injected with BrdU for eight days (FIG. 2E, right panel).

Since IFN-γ enhanced neurogenesis in nine-month-old IFN-γ Tg mice, its effect on neurogenesis was examined in a mouse model of AD, in which Aβ is accumulated initially in the DG. Amyloid precursor protein (APP) Tg mice were crossed with IFN-γ Tg mice, and the double transgenic F1 mice were examined for the amounts of neuronal progenitors generated in the DG. FIG. 3A demonstrates the accumulation of Aβ in the hippocampus of nine-month-old APP Tg mice along with microglial activation at sites of Aβ plaques. The amounts of BrdU/DCX-immunolabeled cells in the DGs of APP Tg mice—but not APP/IFN-γ Tg mice—were lower than those in control wild-type mice (FIG. 3B). Stereological quantification of BrdU, DCX, and BrdU/DCX cells at nine months of age revealed significantly higher amounts of immunolabeled cells in the DGs of APP/IFN-γ Tg mice than in the DGs of APP Tg mice (FIG. 3C). The amounts of BrdU/DCX cells in APP/IFN-γ Tg mice were similar to those in age-matched wild-type mice but lower than those in IFN-γ Tg mice (FIG. 2D). The neurogenesis rate in the DG dropped significantly with age: by three months of age it had already fallen by 65% in wild-type mice, and by nine months it had fallen by 78% in wild-type mice and by 92% in APP Tg mice (FIG. 3D). In parallel, the effect of IFN-γ on the amounts of BrdU/DCX cells generated in the DG became more pronounced, increasing by 1.3- and 1.6-fold in three- and nine-month-old IFN-γ Tg mice, respectively, and by 2-fold in nine-month-old APP/IFN-γ Tg mice (FIG. 3D).

### Example 3

### Oligodendrogenesis is Reduced in IFN-γ Tg Mice

Since the expression of IFN-γ in the brain increased the amounts of BrdU and BrdU/DCX cells in the DGs of IFN-γ and APP/IFN-γ Tg mice (FIGS. 2 and 3), the effect of IFN-γ on increasing the amounts of other neural progenitors or promoting NPC differentiation to neurons while compromising the amount of oligodendrocyte progenitors was studied. Sagittal brain sections were taken from nine-month-old wild-type, IFN-γ Tg, APP Tg, and APP/IFN-γ Tg mice injected with BrdU and immunolabeled for BrdU and NG-2 as described in Methods. Confocal microscopy images from the hippocampus were analyzed for BrdU/NG-2 double-positive cells (FIG. 4A, see arrows). As shown in FIG. 4B, costaining with anti-CD11b (green) and anti-NG2 (red) revealed that the NG2 antibodies specifically labeled oligodendrocyte precursors and not microglia. Three-dimensional projections were generated from Z-stack images of positive cells using Volocity software to confirm the quantification of BrdU/NG2 double-positive cells (FIG. 4C). Stereological analysis revealed that the amount of oligodendrocyte progenitors was significantly higher in nine-month old APP Tg mice than in age-matched wild-type controls (FIG. 4D). Oligodendrogenesis was, however, slightly down-regulated in IFN-γ Tg mice and did not increase in APP/IFN-γ Tg mice (FIG. 4D). It therefore appears that IFN-γ shifts the balance in the hippocampus from oligodendrogenesis to neurogenesis.

### Example 4

### Synaptophysin Reactivity in the Hippocampus of IFN-γ and APP Tg Mice

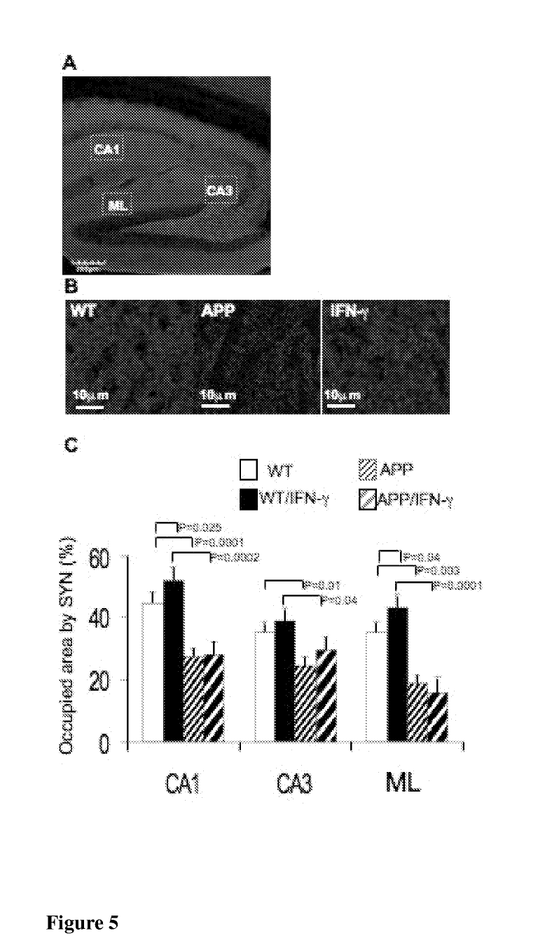

Because IFN-γ is a proinflammatory cytokine that may cause neuronal toxicity, the question of whether the enhanced neurogenesis induced by IFN-γ was a result of neurotoxicity was studied. Since it appears that the concentration of synaptophysin—a protein located at the synapse—is correlated to synaptic activity³⁴, the amounts of synaptophysin in relatively highly innervated regions in the CA1, CA3, and the DG molecular layer of the hippocampus were measured (FIG. 5A). Synaptophysin immunostaining was performed in wild-type, IFN-γ, APP, and APP/IFN-γ Tg mice at nine months of age. Confocal microscopy images were taken from at least three sagittal sections from each brain, and stereological quantification was performed using Volocity software. Compared with the immunofluorescence in wild-type mice, reduced synpatophysin immunofluorescence was observed in the DGs of APP but not IFN-γ Tg mice (FIG. 5B). Quantification analysis demonstrated that synaptophysin immunoreactivity in the CA1, CA3, and the DG was higher in IFN-γ Tg mice than in wild-type controls but remarkably lower in APP and APP/IFN-γ Tg mice (FIG. 5C). In APP and APP/IFN-γ Tg mice, this reduced synaptophysin immunoreactivity was greater in the DG molecular layer than in the CA1 and CA3 regions (FIG. 5C), presumably due to the accumulation of Aβ primarily in the DG at nine months of age (FIG. 3A). Overall, IFN-γ appears to have a neuroprotective effect in aged mice, with the exception of the DG molecular layer of APP Tg mice, where glial activation by Aβ may be the dominant neurotoxic effect.

### Example 5

### Effects of IFN-γ on Spatial Learning and Memory

As shown in FIG. 2, neurogenesis was already significantly lower in the DG at three months than at one month of age. The association of the slower decline in neurogenesis observed in IFN-γ Tg mice at three months with improved spatial learning and memory performance was studied. Three-month-old mice were tested in a Morris water maze (MWM) in the acquisition, probe trial, and reversal phases, as described previously. In the acquisition phase (FIG. 6A), the IFN-γ Tg mice took less time than the controls to find the hidden platform throughout the five-day experiment, with the differences being significant on days 4 and 5 (p<0.05). In the probe trial phase, the IFN-γ Tg mice spent a significantly larger proportion of time than their controls in the quadrant that had previously contained the platform (FIG. 6B, p<0.0015). In the reversal phase, performed over two days for three different platform locations, the IFN-γ Tg group again took significantly less time than the control group to find the hidden platform, primarily on the first day of the new location, with significant differences in values on days 9 and 11 (P<0.05) (FIG. 6C-E). No significant inter-group differences were found between groups in swimming speed (P>0.05) or locomotor abilities (p>0.05).

### Example 6

### Gene Expression Analysis in the Brain of IFN-γ Tg Mice

Limited amounts of IFN-γ expressed in the brain presumably exerted beneficial effects on neurogenesis and neuroprotection. To determine the role of IFN-γ in regulating the immune and neuropoietic milieu in the brain at such low concentrations, qPCR analysis of hippocampus-cortex brain samples from Three-month-old IFN-γ and wild-type control mice were analyzed for gene expression in the brain by qPCR. Brain samples were analyzed according to three categories of genes: 1) genes associated with antigen presentation; 2) immune mediators and regulators; and 3) neurotrophic factors. For each of the genes analyzed, the relative quantity (RQ) was calculated according to the levels of actin expression in each mouse. The results are presented as average RQs of the expressed genes in IFN-γ Tg compared with wild-type mice. P-values and STE were calculated from five to seven pairs of IFN-γ Tg and wild-type mice using one-tailed Wilcoxon matched pairs test. IFN-γ induced a 3.05- and 1.96-fold increase in the expression of the invariant chain Li and the CD86 costimulatory molecules, respectively, vs. control animals (Table 1A). Among the proinflammatory cytokines, both IL-6 and TNF-α were mildly, but significantly, up-regulated—by 3.18- and 2.65-fold, respectively (Table 1B). Up-regulation of IL-1γ by 2.67-fold was not significant (Table 1B). Intracellular signaling of IFN-γ was regulated in the brain, even in these limited amounts, as indicated by the increased amounts of SOCS1 (×1.96) (Table 1B). The three neuropoietic factors, brain-derived neurotrophic factor (BDNF), insulin-like growth factor (IGF-1), and neurotrophin 3 (NT-3) were up-regulated in IFN-γ Tg mice by 1.97-, 1.7-, and 1.78-fold, respectively, whereas levels of ciliary neurotrophic factor (CNTF) were similar in IFN-γ Tg and wild-type mice. Expression of LIF, a major player in astrogenesis, was down-regulated by 1.42-fold compared with wild-type controls (Table 1C).

TABLE 1
Quantitative PCR a nalysis of immune and neurotrophic
factors in the brains of IFN-γ Tg mice.

| Gene | RQ ± STE | P-value |
|---|---|---|
| A. Antigen presentation | | |
| Invariant chain (Li) | 3.05 ± 1.52 | 0.0391 |
| CD86 | 1.96 ± 0.64 | 0.0078 |
| B. Immune mediators/regulators | | |
| IL-6 | 3.18 ± 2 | 0.0286 |
| IL-1β | 2.67 ± 0.9 | NS |
| TNF-α | 2.65 ± 0.27 | 0.0313 |
| TGF-β | 1.8 ± 0.71 | NS |
| GM-CSF | Not detected | |
| SOCS 1 | 1.96 ± 0.41 | 0.0313 |
| SOCS 2 | 1.43 ± 0.2 | NS |
| SOCS 3 | No difference | |
| C. Neurotrophic factors | | |
| CNTF | No difference | |
| NT-3 | 1.78 ± 0.36 | 0.0391 |
| BDNF | 1.97 ± 0.41 | 0.0313 |
| IGF-1 | 1.7 ± 0.29 | 0.0078 |
| LIF | 0.7 ± 0.12 | 0.0625 |

Three-month old IFN-γ and wild-type control mice were analyzed for gene expression in the brain by qPCR as described in Methods. For each of the genes analyzed, the relative quantity (RQ) was calculated according to the levels of actin expression in each mouse. The results are presented as average RQs of the expressed genes in IFN-γ Tg compared with wild-type mice. P-values and STE were calculated from five to seven pairs of IFN-γ Tg and wild-type mice using one-tailed Wilcoxon matched pairs test.

### Example 7

### Accumulation of Amyloid Beta in the Brain

Amyloid-β load is characterized by diffuse and senile plaques. Senile plaques contain fibrillary form of Aβ with activated microglia and astrocytes surrounding it, while diffuse plaques usually consist of non-fibrillary forms and few activated glia cells. Accumulation of plaques in the DG and cortex area was examined by immunostaining of Aβ1-42 antibody. In young APP transgenic mice (3-month-old) amyloid plaques were undetectable (FIG. 7a). Immunolabeling of brain sections from adult mice (9-month-old) revealed intense staining of compact senile plaques and more lightly diffuse plaque deposits (FIG. 7b). Plaques were more abundantly present in 16-month-old transgenic mice and appeared more condensed and larger in size (FIG. 7c). Comparison of immunoreactivity to Aβ between APP transgenic (Tg) and control mice showed that Aβ accumulated only in transgenic mice. No detectable Aβ staining was observed for 3-, 9-, and 16-month-old WT mice (data not shown).

### Example 8

### Activation of the Innate Immune System

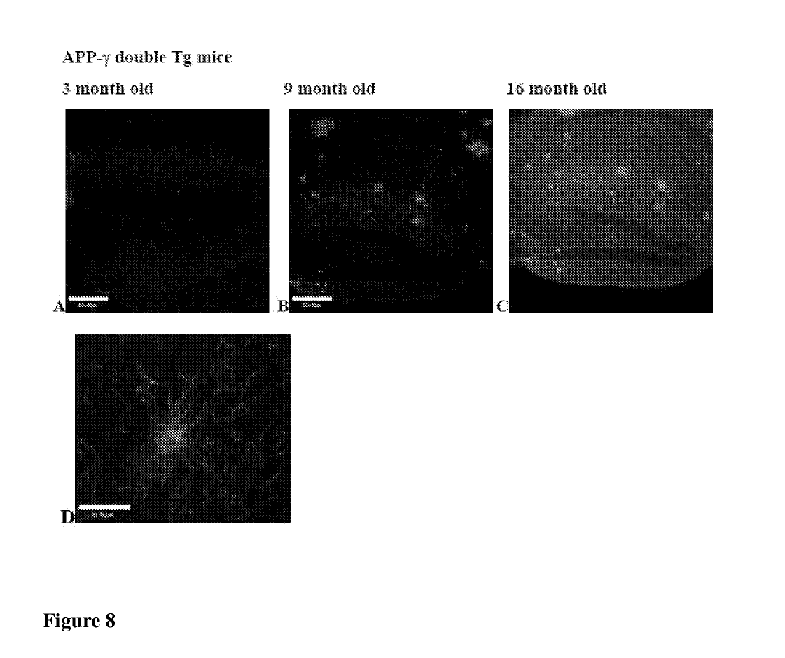

Oligomerization of Aβ1-42 and its deposition as senile and diffuse plaques in the brain lead to microglial and astrocyte activation. Microglia are the immune cells of the central nervous system (CNS) which become chronically activated in the context of Alzheimer's disease. Activation of microglia was evaluated by CD11b staining, an integrin which is over-expressed in activated microglia. CD11b staining was not detected in 3-month-old WT controls and APP Tg mice (FIG. 8a). Nine-month-old APP Tg mice showed increased immunoreactivity of CD11b. These cells were detected in the hippocampus and the cerebral cortex, areas which are mainly affected in AD (FIG. 8b). In 16-month-old APP Tg mice a substantial activation of microglia was observed in the hippocampus and the cortex (FIG. 8c). Larger magnification of the clustered area showed colocalization between Aβ deposits and CD11b cells highly ramified (FIG. 8d).

### Example 9

### Astrocyte Activation

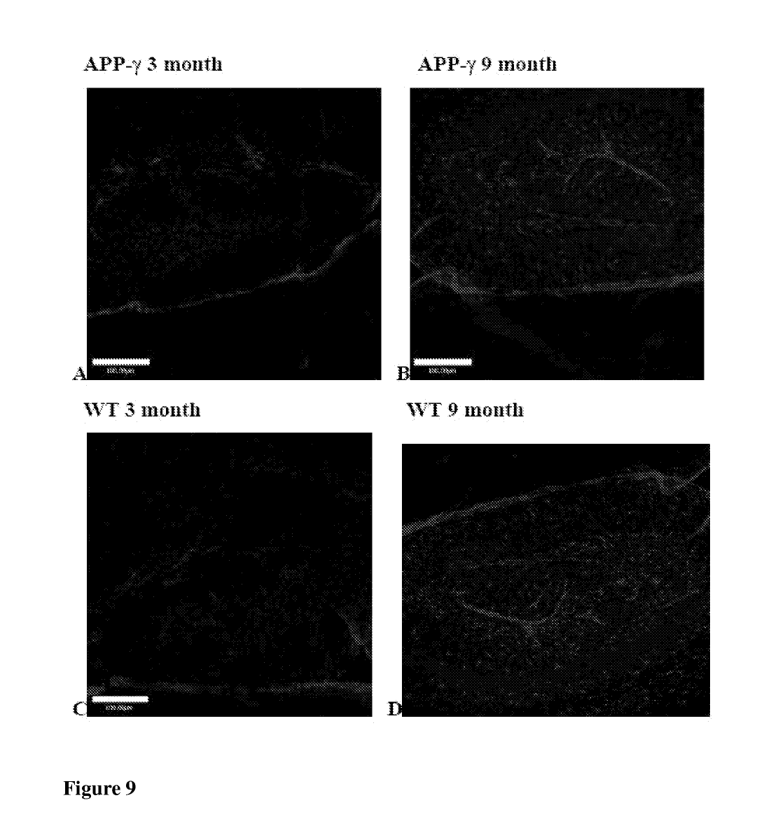

Astrocyte activation was observed using glial fibrillary acidic protein (GFAP) antibody. Glial fibrillary acidic protein (GFAP)-immunopositive astrocytes were observed in WT and 3-month-old APP Tg mice (FIG. 9a-b). More intense staining was observed in 9-month-old a WT and APP Tg mice, with the majority of GFAP-positive foci randomly distributed within the cortex and hippocampus for both WT and APP Tg (FIG. 9a-d).

### Example 10

### Neurogenesis is Decreased in DG of Aged Mice

Adult hippocampal neurogenesis originates from precursor cells in the adult DG and results in new granule cell neurons. Two parameters were assessed to characterize the regulation of adult neurogenesis: proliferation in the subgranular zone (SGZ) and cell survival. To detect proliferating cells mice were injected with BrdU, a synthetic thymidine analog that is incorporated into the DNA of proliferating cells. BrdU persists in daughter cells and can be detected by immunolabeling at different intervals after the injection. During the proliferation stages the cell expresses doublecortin (DCX) protein, microtubule-associated protein, which is a distinct marker of newly differentiated and migrating neurons. It is expressed in mitotic stage and in early post mitotic phase, between 1 to 7 days post BrdU injection. NeuN, neuron-specific nuclear protein, is specifically expressed by post mitotic immature and mature differentiated neurons and the earliest time point it could be observed is 4 days post-BrdU injection.

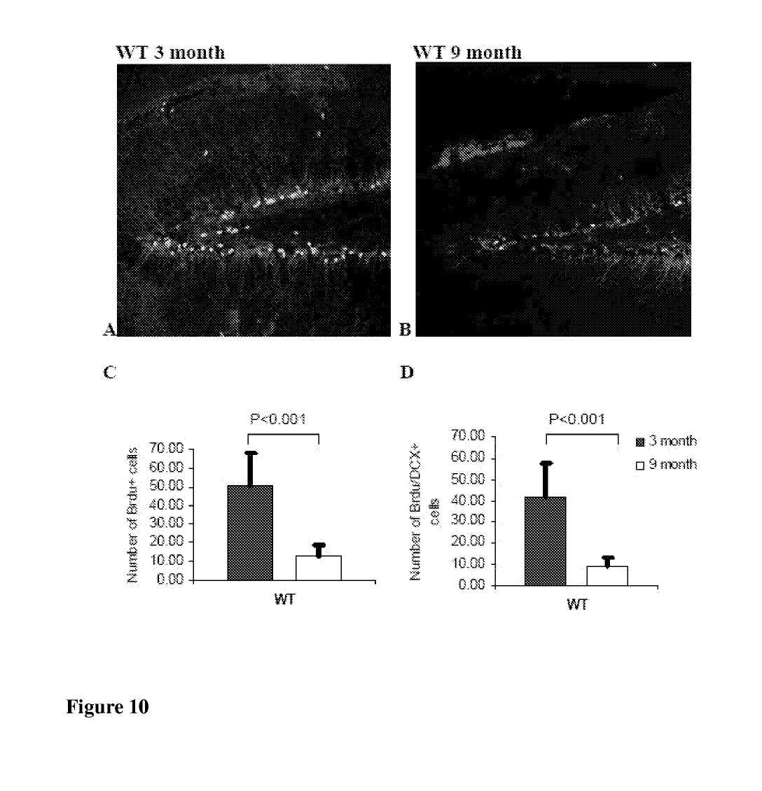

The proliferation phase of neurogenesis in young (3-month-old) and adult (9-month-old) mice was detected by injecting BrdU for eight consecutive days and sacrificing the mice a day later. The labeled cells represented all types of proliferating cells including glia cells and neurons. Double labeling with DCX/BrdU was conducted to further establish the phenotype of these cells. The tissues were stained for DCX (red) and BrdU (green); double-labeled cells appear in yellow. Animals received daily intraperitoneal injection of BrdU (50 mg/g of body weight per day) for 8 days to label dividing cells. Mice were sacrificed a day later and perfused with paraformaldehyde (4%) and transferred to sucrose (30%) to achieve cryoprotection. IHC staining was conducted in free-floating sections cut into 30 μm by cryostat. Antibodies were incubated overnight (4° C.) with α-DCX (1:50) and α-BrdU (1:200) followed by biotin α-goat (1:400) and streptavidin 546 (1:500) and α-rat Alexa488 (1:500). Five animals and at least four tissues throughout the hippocampus of each mouse were taken for quantification of proliferating cells. Cells were counted in the DG area (granular and subgranular zone) of each mouse and the average number of cells was calculated. BrdU-labelled nuclei were counted and found to decrease by 74% in the brains of old vs. young WT mice (FIG. 10a). When double-labeled DCX/BrdU cells were counted, a decrease of 79% was observed, further indicating an age-related decline in neurogenesis (FIG. 10b). IHC staining with DCX and BrdU showed similar phenomena of a decrease in proliferative neurons in the DG with age (FIG. 10c, d).

### Example 11

### Neurogenesis is Decreased in the DG of APP Tg Mice

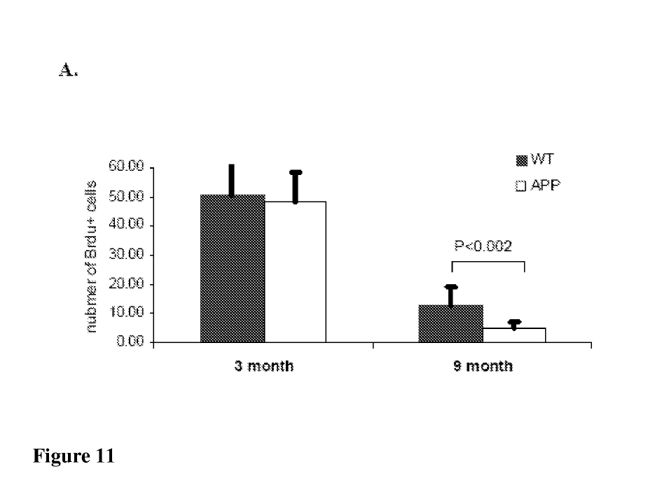

The number of proliferative cells and newly differentiated cells between WT and APP Tg mice was compared to examine the effect of Alzheimer's progression on neurogenesis in a PDGF-APP mouse model. The neurogenesis level was almost identical between WT and APP Tg in 3-month-old mice, prior to Aβ plaque formation. The average number of BrdU cells in the DG area per brain section of 30 μm was 50±17 for WT mice and 48±10 for APP Tg mice (FIG. 11a). However, at 9 months, when APP Tg mice exhibited senile and diffused plaques, a significant decrease in neurogenesis was observed compared to WT. The number of BrdU stained cells in the DG area per brain section of 30 μm was 5±2 compared to 12±6 in WT (FIG. 11a, right panel). Similar results were obtained upon counting the newborn neuron cells double-labeled with antibodies to DCX and BrdU. The average number of cells in the DG area per brain section per 30 μm was 9±4 for WT mice and 4±1.2 for APP Tg mice. Thus, the data showed that the level of proliferating neurons was 55% less in APP Tg compared to WT mice.

### Example 12

### Effect of IFN-γ on Neurogenesis

IFN-γ is a key T-cell derived cytokine that induces microglia differentiation to APC and thus potentially stimulates a dialog with T-cells. In the mouse model described herein, IFN-γ is expressed in the brain under the transcriptional control of the myelin basic protein (MBP) gene. Previous studies have shown that although IFN-γ is a pro-inflammatory cytokine, it can also induce neuroprotection. In vitro, IFN-γ supports differentiation of neuron precursor cells toward a phenotype of newly born neurons.

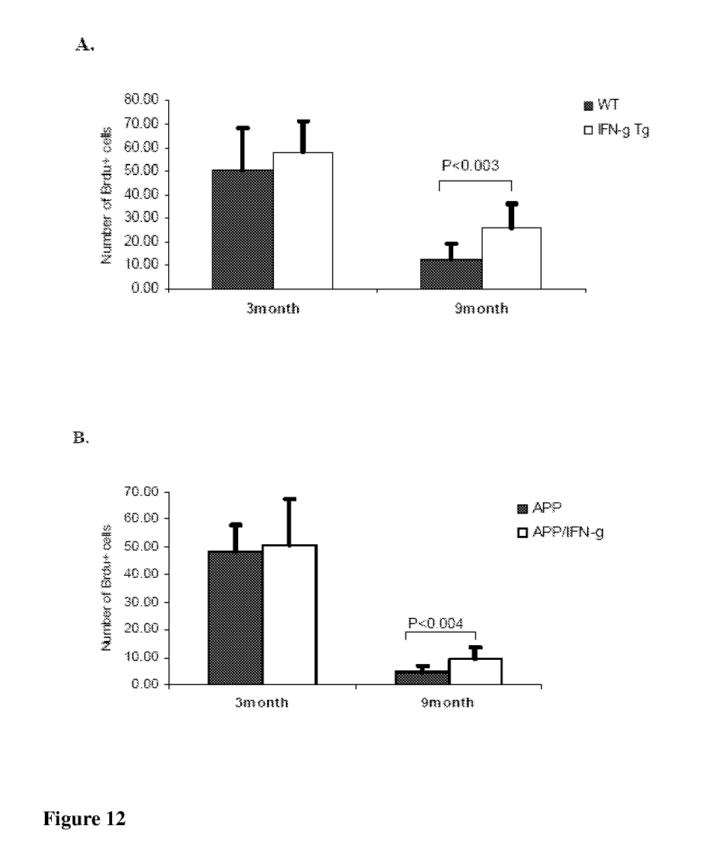

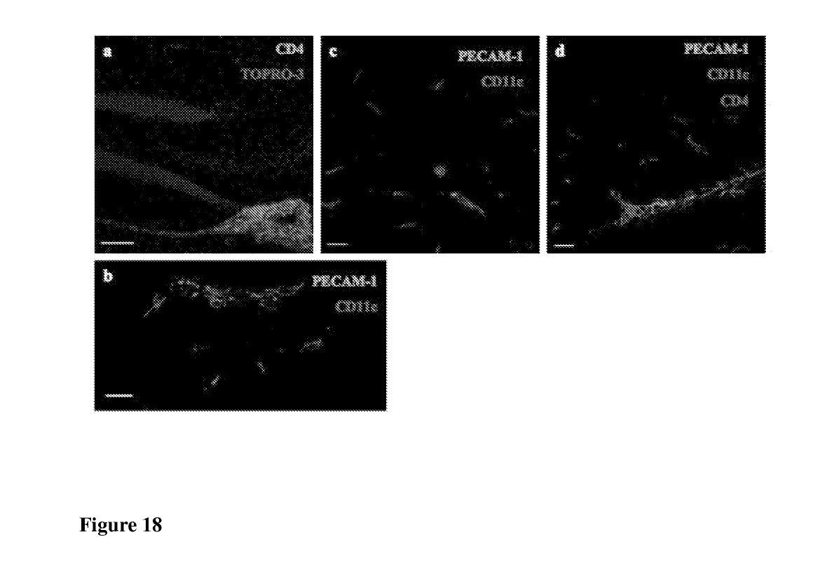

The ability of IFN-γ to induce neurogenesis under in vivo conditions was examined by injecting mice with BrdU for 8 consecutive days and sacrificing the mouse 1 day later. Using IHC techniques, tissues were stained with DCX and BrdU and images of the DG area were taken with a confocal microscope. The comparison was first done in Tg IFN-γ mice vs. wild-type (WT) mice. In 3-month-old mice, the average number of BrdU cells in the DG area per brain section of 30 μm did not differ significantly between the two groups (WT 58±13, IFN-γ Tg mice 65±15.3, FIG. 12a). A possible trend towards elevation in neurogenesis could be observed in IFN-γ Tg compared to WT mice. However, 9-month-old mice showed a significant difference which is up to 2-fold increase of BrdU+ cells comparing IFN-γ Tg and WT mice (WT 12.8±6.14, IFN-γ 25.5±10.7, FIG. 18a). Similar results were obtained for newborn neurons double-labeled with DCX and BrdU antibodies (data not shown). In addition, the capacity of IFN-γ to ameliorate the decline in neurogenesis detected in APP Tg mice was examined. APP mice were crossed with IFN-γ Tg mice and compared to APP Tg mice. The effect of IFN-γ in these mice was similar to the effect in the non-APP Tg mice. That is, BrdU and BrdU/DCX+ cells in the DG of 3-month-old APP and APP/IFN-γ Tg mice did not show significant difference (BrdU+ cells: APP Tg 48.3±9.9, APP/IFN-γ 50.9±16.3; DCX/BrdU+ cells: APP Tg 38.5±10, APP-IFN-γ 39.4±13.7 FIG. 12B). However, compared with APP 9-month-old mice, APP/IFN-γ showed a 2-fold increase in BrdU and BrdU/DCX+ cells in the DG area per brain section of 30 μm (BrdU+ cells: APP Tg 4.8±2.1, APP/IFN-γ 9.2±4.5; DCX/BrdU+ cells: APP 3.56±1.1, APP-IFN-γ 6.3±2.1 FIG. 12B). The significant increase in proliferation at the DG of APP/IFN-γ double Tg mice show recovery to the value observed in WT mice at the same age.

### Example 13

### Effect of IFN-γ on Oligodendrogenesis

The Tg mice described herein showed an elevated level of proliferated precursor and newly differentiated neurons induced by IFN-γ cytokine. It is important to determine whether the specificity of this effect for the neuron lineage or whether oligodendrocytes are also influenced. In vitro, microglia activated by low level of IFN-γ showed bias towards neurogenesis, while IL-4 induced oligodendrogenesis.

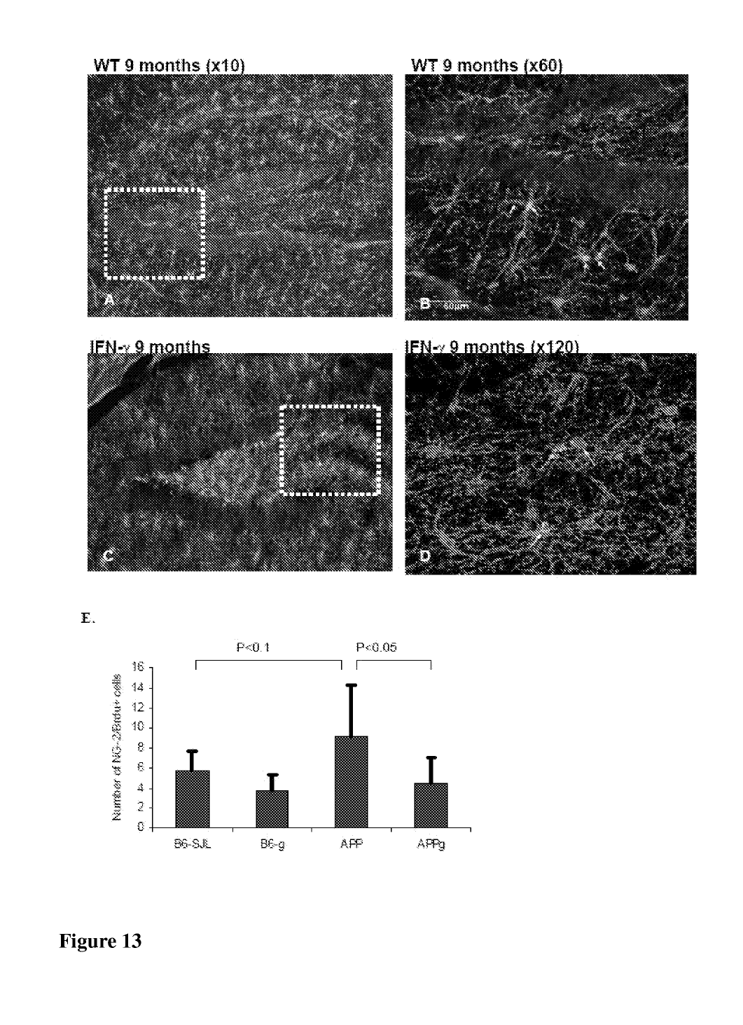

Oligodendrocytes in 9-month-old mice were counted by staining with NG-2, a membrane chondroitin sulfate proteoglycan, which is found on surface of oligodendrocyte precursor cells. The DG area was imaged by confocal microscope and virtual z-stack sections were taken. Cells stained with BrdU were counted within the DG (granule and subgranular zones) and surrounding the DG area, towards the hippocampus. To identify newborn oligodendrocytes, cells were double-labeled with NG-2/BrdU and counted in the same procedure. A decrease in oligodendrogenesis was observed in both IFN-γ and APP/IFN-γ double Tg mice compared to mice that did not express IFN-γ in the brain (FIG. 13a: WT 5.7±1.9, IFN-γ 3.7±1.6; APP 9.2±5, APP/IFN-γ-4.5±2).

### Example 14

### Aβ Immunization Results in Immune Cell Infiltration into the CNS

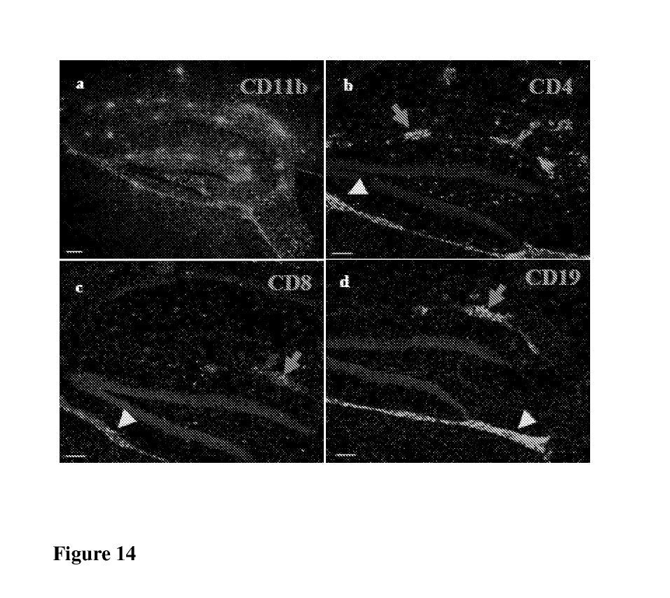

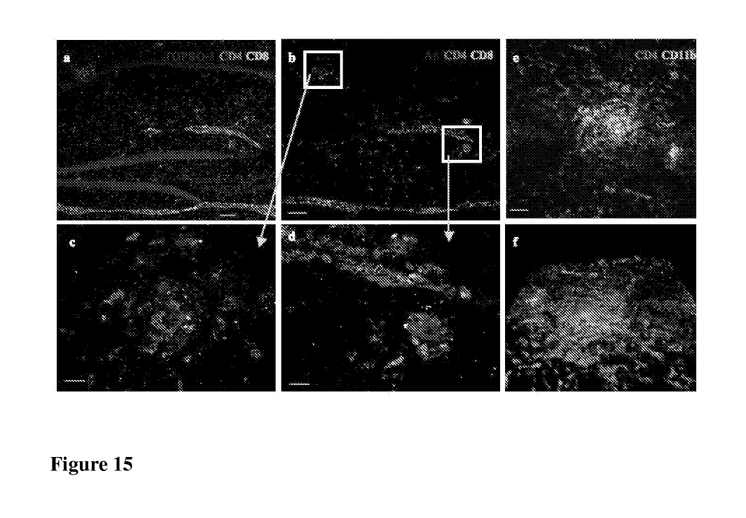

Ten-month-old transgenic APP/IFN-γ mice were immunized by one subcutaneous injection of 100 micrograms of peptide. with amyloid β protein (Aβ) and sacrificed 19 days later. Brains were excised, embedded in OCT and frozen. Slices were cut using a cryostat and stained with specific fluorescent-labeled antibodies against CD11b resident microglia and infiltrating monocytes, CD4 T cells, CD8 T cells and CD19 B cells. Immunization with Aβ resulted in the infiltration of monocytes (FIG. 14a, green), CD4 T cells (FIG. 14b, green), CD8 T cells (FIG. 14c, green) and CD19 B cells (FIG. 14d, green). Infiltrates were observed in parenchymal vessels (FIGS. 14b, c, d, red arrows). Meningeal infiltration was also observed (FIGS. 14c, d, yellow arrows).

### Example 15

### T cells Cross into the Parenchyma and Migrate to Aβ Plaques

Mice were treated as described in Example 14, above. Tissue sections were stained with fluorescent-labeled antibodies against for CD4 (red) and CD8 (green) cells, and counter-stained with TOPRO-3, a stain specific for nucleic acids. (blue) (FIG. 15a) or Aβ (FIG. 15b). FIGS. 15c and d show higher magnifications of amyloid plaques targeted and stained by both CD4 and CD8 T cells (yellow staining). FIG. 15e shows a section stained for CD4 (red), CD11b (green) and Aβ (blue) and reveals infiltration of T cells into the parenchyma and targeting of amyloid plaques.

### Example 16

### Up-regulation of Adhesion Molecules Following Aβ Vaccination of APP/IGN-γ

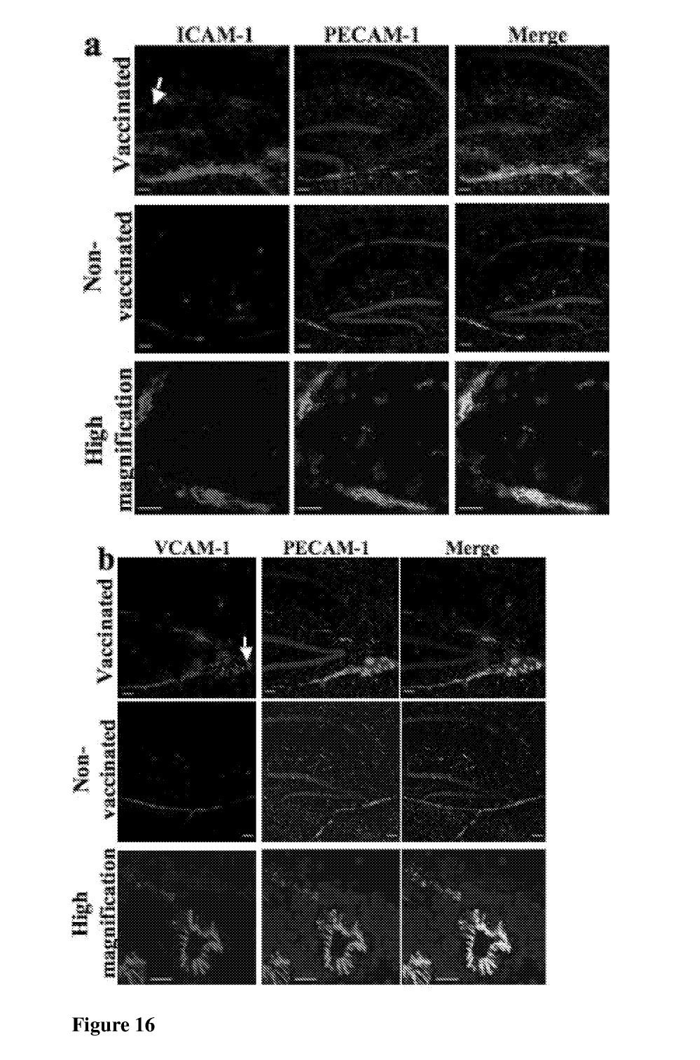

Ten-month-old APP/IFN-γ mice were immunized with Aβ/CFA or left untreated, and killed 19 days later. FIG. 16a shows brain sections were stained for ICAM (intracellular adhesion molecule)-1 (red) and PECAM (platelet/endothelial adhesion molecule)-1 (green) and counter-stained with TOPRO-3 (blue). FIG. 16a shows brain sections were stained for VCAM (vascular cell adhesion molecule)-1 (red) and PECAM-1 (green) and counter-stained with TOPRO-3 (blue). FIG. 16c shows quantitative analysis of ICAM-1 and VCAM-1 in parenchymal and meningeal blood vessels in the brain. A significant up-regulation of ICAM-1 in vaccinated mice was observed.

### Example 17

### CD11c+ Dendritic Cells Localize to the Perivascular Area Following Aβ Vaccination

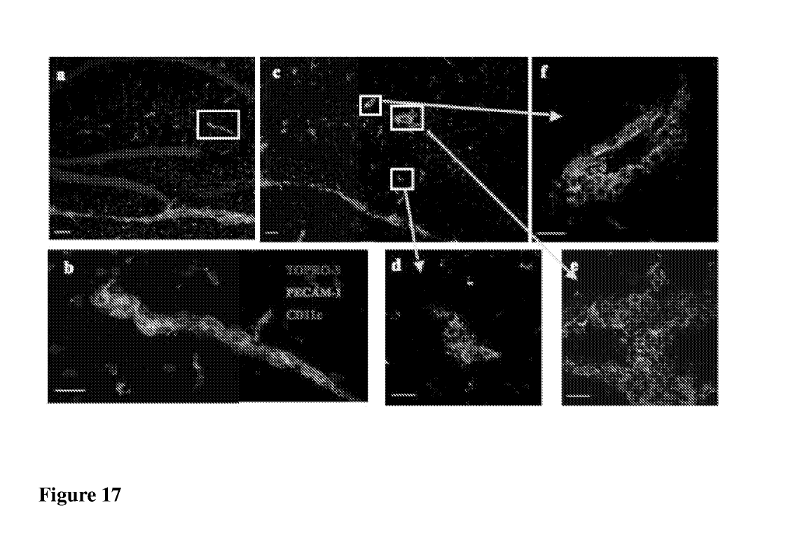

Transgenic APP/IFN-γ mice were vaccinated with Aβ/CFA and sacrificed 13 (FIG. 17a) or 17 (FIG. 17b) days later. Brain sections were stained for PECAM-1 (green), CD11c (red) and counter-stained with TOPRO-3 (a,b) (blue) or CD4 (FIG. 17c, d, e, f) (blue). Following Aβ vaccination of APP/IFN-γ mice, CD11c+dendritic cells localized to the perivascular area and were in contact with infiltrating CD4 T cells.

### Example 18

### Effect of PLP Vaccination in APP/IFN-γ Transgenic Mice

Nine-month-old transgenic APP/IFN-γ mice were vaccinated with PLP (proteolipid protein)/CFA (complete Freund's adjuvant) and sacrificed 19 days later. Brain sections were stained with fluorescent-labeled antibodies against CD4 and counter-stained with TOPRO-3 (FIG. 18a). Staining with fluorescent-labeled antibodies against CD11c (red) and PECAM-1 (green) (FIGS. 18b, c) revealed localization of CD11c dendritic cells to blood vessels in the parenchyma. Triple staining with fluorescent-labeled antibodies against CD11c (red), PECAM-1 (green) and CD4 (blue) revealed localization to blood vessels in the meninges and contact with meningeal CD4 T cells (FIG. 18d).

### Example 19

### Aβ Immunization Results in BDNF Secretion by Glia Cells

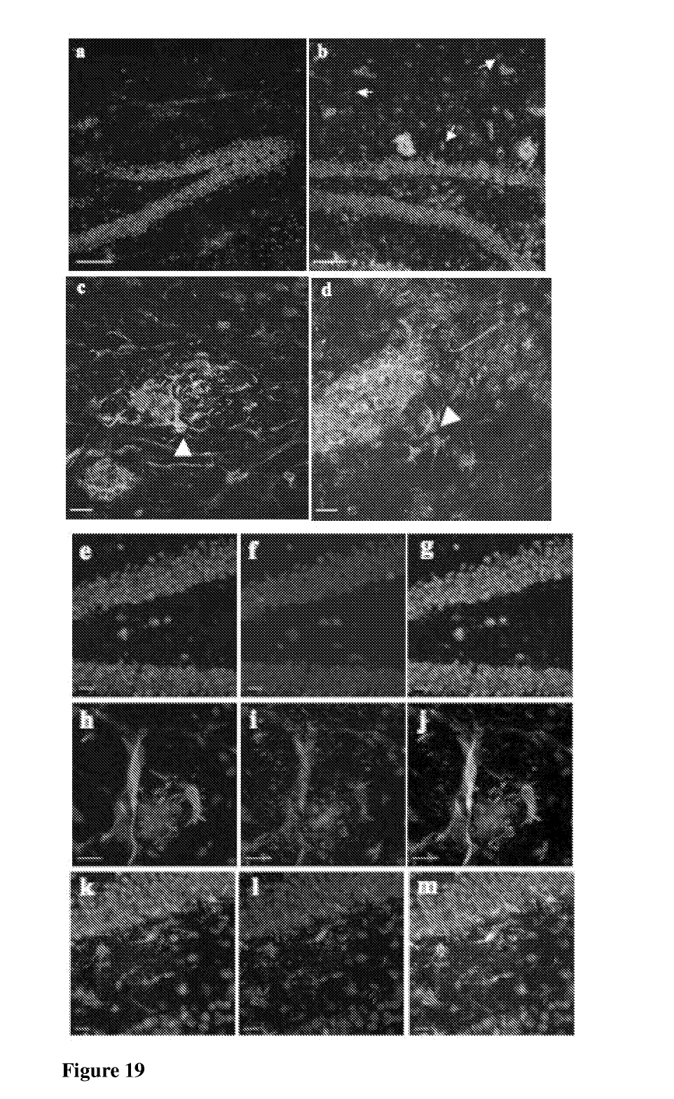

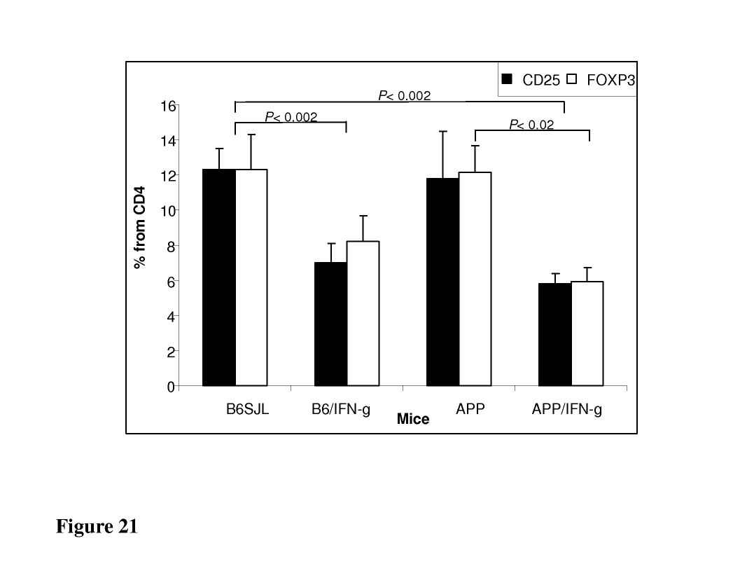

Mice were immunized with Aβ and brain sections were stained with fluorescent-labeled antibodies against BDNF (green) and CD4 (red) (FIGS. 19a, b, c, d). FIGS. 19c and d show areas of sections from immunized mice in which interaction between CD4 T cells and BDNF-expressing glial cells, marked with white arrows, can be observed. Sections showing staining with BDNF (green) (e) and NewN (red) (f) are also presented, with a merged image in (g). FIGS. 21h, i, k and 1 show brain sections of GFAP (red)-stained astrocytes, further stained with BDNF (green), with the merged images in FIGS. 21j and m.

### Example 20

Prevalence of CD4+CD25+ and CD4+FOXP3+ T cells in Aged IFN-γ Tg Mice

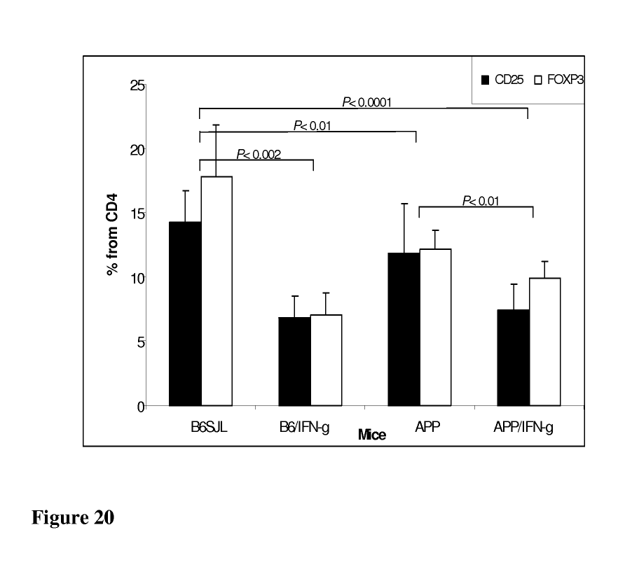

In order to determine whether brain over-expression of IFN-γ in aged mice affects the prevalence of peripheral Tregs, spleen cells from B6SJL, B6SJL\INF-γ, APP and APP/IFN-γ mice at 9-11 months of age were probed for CD4, CD25 and FOXP3 expression by flow cytometry. In B6SJL/IFN-γ and APP/IFN-γ mice, significantly fewer CD4+ T cells were also positive for CD25+ and FOXP3+ (6.8, 7.4 and 7 and 9.8% for CD25 and FOXP3, respectively) as compared to control mice (14.3 and 17.7% for CD25 and FOXP3, respectively) (FIG. 20). Surprisingly, the proportion of CD25+ and FOXP3+ expressing cells among CD4+ T cells from APP mice was also significantly lower compared with the control (11.8 and 12.1% for CD25 and FOXP3, respectively), though demonstrably greater than in IFN-γ Tg mice (FIG. 20). These results indicate that lower frequencies of Tregs are found in APP mouse model compared with control, while IFN-γ expression in transgenic mice further reduced the number of cells.

### Example 21

### CD4+CD25+ Treg Numbers are Influenced by Aging, AD and IFN-γ

The proportion of CD4+CD25+ and CD4+FOXP3+ T cells from the spleens of 2-4 month old mice was analyzed. Spleen cells of B6SJL, B6 μL\IFN-γ, APP and APP/IFN-γ mice were stained and analyzed as mentioned previously. Similar to the elderly mice, in B6SJL/IFN-γ and APP/IFN-γ Tg mice, lower numbers of CD4+CD25+ and CD4+FOXP3+ were detected among CD4+ T cells compared with control (7, 5.8 and 12.3 and 8.2, 5.9 and 12.3% for CD25 and FOXP3, respectively) (FIG. 21). However, contrary to the aged APP mice, which showed a significant decrease in Tregs compared with the control, there was no significant difference between the number of Tregs from young APP and young controls (FIG. 21). The frequency of CD4+FOXP3+ among CD4+ T cells was significantly increased with age, except for CD4+CD25+ Tregs from APP, which remained unchanged. CD4+CD25+ cells showed a trend of higher frequency with aging, although this trend was not significant.

### Example 22

### Treg Effects on Spatial Learning and Memory

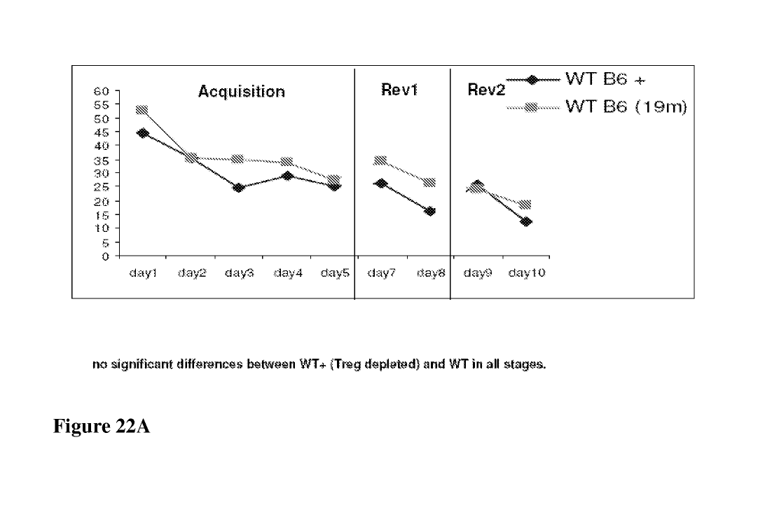

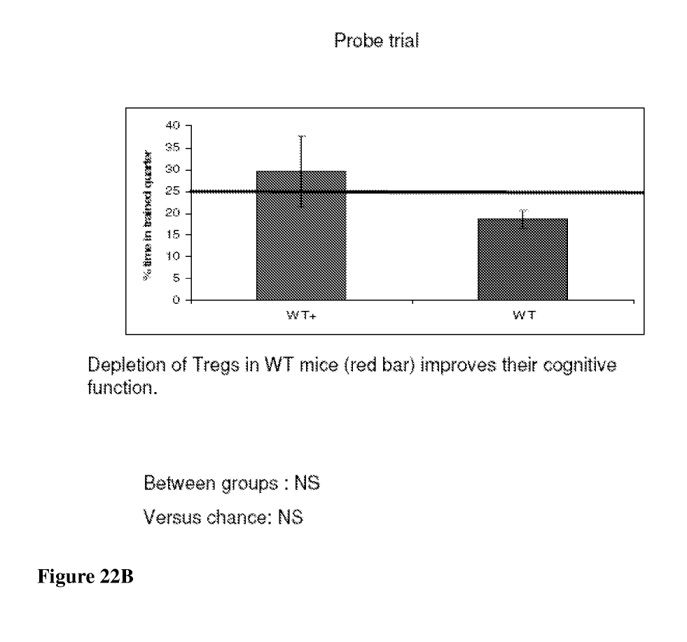

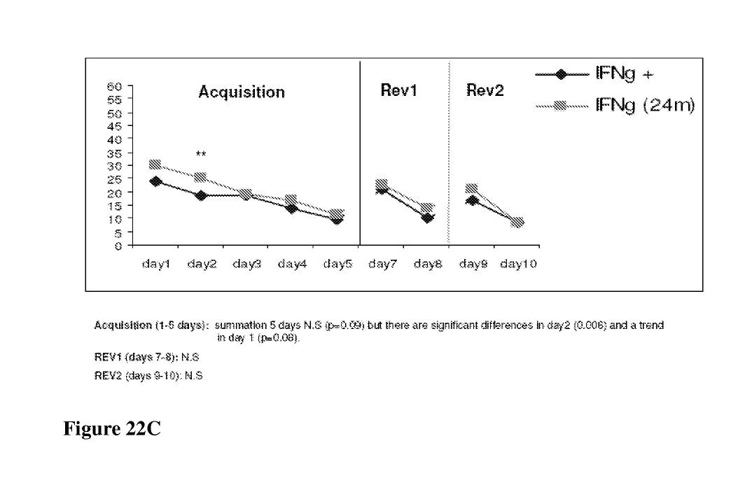

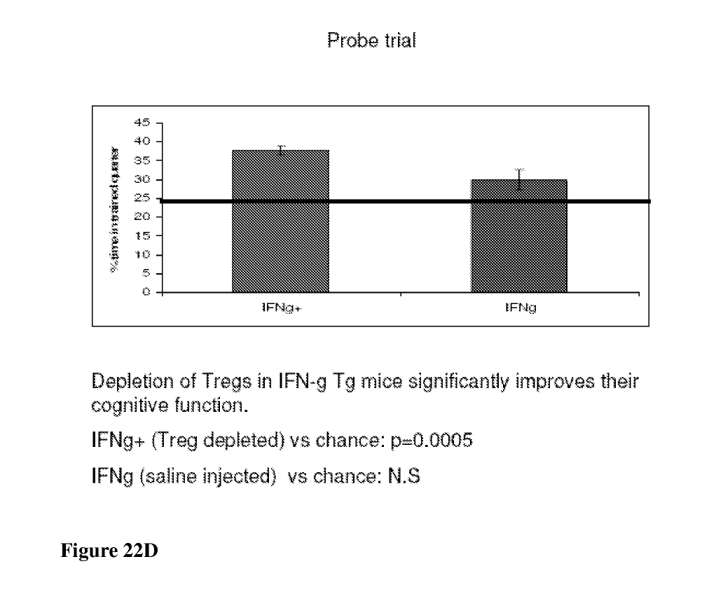

The effect of T regulatory (Treg) cell depletion on spatial learning and memory performance was determined. Three-month-old mice were tested in a Morris water maze (MWM) system as described hereinabove, during the acquisition, probe trial, and reversal phases. In the acquisition phase, no significant differences were recorded throughout the five-day experiment between Treg-depleted wild-type and wild-type mice (FIGS. 22A & B), nor were there significant differences in the reversal phase, performed over two days. Significant differences in acquisition rates between young and old (24-month-old) IFN-γ Tg mice were recorded on day 2 of the acquisition phase (p=0.006), with a trend observed on day 1 (p=0.08) (FIG. 22C). Depletion of Tregs in IFN-γ Tg mice was shown to significantly (p=0.0005) improve their cognitive function compared to non-Treg-depleted IFN-γ Tg mice, as demonstrated by the percent time spent in the trained quadrant (FIG. 22D).

1-59. (canceled)

60. A method for treating or preventing a neurodegenerative disease in a subject in need thereof, comprising:
(a) administering to said subject, in a brain specific manner an agent which increases brain levels of interferon-γ, wherein said agent is selected from the group consisting of IFN-γ, a nucleic acid encoding IFN-γ and T-cells; and
(b) peripherally administering to said subject an agent which reduces the number of T regulatory (Treg) cells, wherein said agent is selected from the group consisting of an anti-CD25 antibody, an anti-Foxp3 antibody or a combination thereof,
thereby treating or preventing said neurodegenerative disease.

61. The method of claim 60, wherein said agent which increases brain levels of interferon-γ is interferon-γ.

62. The method of claim 60, further comprising administering an agent which suppresses neurotoxic inflammatory brain responses.

63. The method of claim 60, wherein said agent of (a) comprises a liposome.

64. The method of claim 60, further comprising administering neurotrophins.

65. The method of claim 64, wherein said neurotrophins comprise BDNF, NT-3 or NT-4.

66. The method of claim 60, wherein said administration of (a) comprises intracranial administration.

67. The method of claim 60, wherein said neurodegenerative disease or disorder comprises Alzheimer's disease, Parkinson's disease, Huntington's disease, multiple sclerosis, diabetic neuropathy or amyotrophic lateral sclerosis (ALS).

68. A method for treating or preventing a neurodegenerative disease in a subject in need thereof, said method comprising peripherally administering to the subject a an agent which reduces the number of T regulatory (Treg) cells wherein said agent is selected from the group consisting of an anti-CD25 antibody, an anti-Foxp3 antibody or a combination thereof,
thereby treating or preventing said neurodegenerative disease.

69. The method of claim 68, further comprising administering an agent which suppresses neurotoxic inflammatory brain responses.

70. The method of claim 68, wherein said subject has increased brain levels of interferon-γ.

71. The method of claim 68, wherein said neurodegenerative disease or disorder comprises Alzheimer's disease, Parkinson's disease, Huntington's disease, multiple sclerosis, diabetic neuropathy or amyotrophic lateral sclerosis (ALS).

72. A method for inducing or enhancing neurogenesis in a subject in need thereof, said method comprising:
(a) administering to said subject, in a brain specific manner, an agent which increases brain levels of interferon-γ, wherein said agent is selected from the group consisting of IFN-γ, a nucleic acid encoding IFN-γ and T-cells; and
(b) peripherally administering to said subject an agent which reduces the number of T regulatory (Treg) cells wherein said agent is selected from the group consisting of an anti-CD25 antibody, an anti-Foxp3 antibody or a combination thereof,
thereby treating or preventing said neurodegenerative disease.

73. The method of claim 72, wherein said agent which increases brain levels of interferon-γ is interferon-γ.

74. The method of claim 72, further comprising administering an agent which suppresses neurotoxic inflammatory brain responses.

75. The method according to claim 72, wherein said agent of (a) comprises a liposome.

76. The method according to claim 72, wherein said administration of (a) comprises intracranial administration.

77. The method of claim 72, further comprising administering neurotrophins, wherein said neurotrophins comprise BDNF, NT-3 or NT-4.

78. The method of claim 72, wherein said neurodegenerative disease comprises Alzheimer's disease, Parkinson's disease, Huntington's disease, multiple sclerosis, diabetic neuropathy or amyotrophic lateral sclerosis (ALS).

79. A method for treating or preventing a neurodegenerative disease in a subject in need thereof, said method comprising administering to the subject, in a brain specific manner, an agent which increases brain levels of interferon-γ, wherein said agent is selected from the group consisting of IFN-γ, a nucleic acid encoding IFN-γ and T-cells, and wherein said subject is a T regulatory (Treg) depleted subject, thereby treating or preventing said neurodegenerative disease in said subject.

80. The method of claim 79, wherein said agent which increases brain levels of interferon-γ is interferon-γ.

81. The method according to claim 79, wherein said administration of (a) comprises intracranial administration.

82. The method of claim 79, wherein said neurodegenerative disease or disorder comprises Alzheimer's disease, Parkinson's disease, Huntington's disease, multiple sclerosis, diabetic neuropathy or amyotrophic lateral sclerosis (ALS).

83. A kit for treating or preventing a neurodegenerative disease in a subject, comprising:
an agent formulated for brain specific administration which increases brain levels of interferon-γ, wherein said agent is selected from the group consisting of IFN-γ, a nucleic acid encoding IFN-γ and T-cells; and
an agent formulated for peripheral administration which reduces the number of T regulatory (Treg) cells wherein said agent is selected from the group consisting of an anti-CD25 antibody, an anti-Foxp3 antibody or a combination thereof.

84. The kit of claim 83, wherein said agent which increases brain levels of interferon-γ is interferon-γ.

85. The kit according to claim 83, wherein said agent which reduces the number of T regulatory (Treg) cells is anti-CD25 antibody.

86. The kit of claim 83, wherein said neurodegenerative disease or disorder comprises Alzheimer's disease, Parkinson's disease, Huntington's disease, multiple sclerosis, diabetic neuropathy or amyotrophic lateral sclerosis (ALS).
